(19) United States
(12) Patent Application Publication
(10) Pub. No.: US 2021/0220494 A1
(43) Pub. Date: Jul. 22, 2021

(54) COMPOSITIONS AND METHODS FOR TARGETED PARTICLE PENETRATION, DISTRIBUTION, AND RESPONSE IN MALIGNANT BRAIN TUMORS

(71) Applicants: Memorial Sloan Kettering Cancer Center, New York, NY (US); Cornell University, Ithaca, NY (US)

(72) Inventors: Michelle S. Bradbury, New York, NY (US); Michael Overholtzer, New York, NY (US); Cameron Brennan, Haworth, NJ (US); Barney Yoo, New York, NY (US); Jedd D. Wolchok, New York, NY (US); Ulrich Wiesner, Ithaca, NY (US)

(21) Appl. No.: 16/095,780

(22) PCT Filed: Apr. 28, 2017
(86) PCT No.: PCT/US17/30056

§ 371 Date: Oct. 23, 2018

Related U.S. Application Data
(60) Provisional application No. 62/330,029, filed on Apr. 29, 2016.

(51) Int. Cl. A61K 51/12; A61K 47/69; A61P 35/00; A61K 51/04

(52) U.S. Cl. CPC A61K 51/1244; A61K 47/6929; A61K 2121/00; A61K 47/6923; A61K 51/0474; A61P 35/00

(57) ABSTRACT

Described herein are nanoparticle conjugates that demonstrate enhanced penetration of tumor tissue (e.g., brain tumor tissue) and diffusion within the tumor interstitium, e.g., for treatment of cancer. Further described are methods of targeting tumor-associated macrophages, microglia, and/or other cells in a tumor microenvironment using such nanoparticle conjugates. Moreover, diagnostic, therapeutic, and theranostic (diagnostic and therapeutic) platforms featuring such nanoparticle conjugates are described for treating targets in both the tumor and surrounding microenvironment, thereby enhancing efficacy of cancer treatment. Use of the nanoparticle conjugates described herein with other conventional therapies, including chemotherapy, radiotherapy, immunotherapy, and the like, is also envisaged.

### CROSS REFERENCE TO RELATED APPLICATION

This application claims the benefit of U.S. Application Ser. No. 62/330,029 filed on Apr. 29, 2016, the disclosure of which is hereby incorporated by reference in its entirety.

### GOVERNMENT SUPPORT

This invention relates was made with government support under grant number CA199081 awarded by National Institutes of Health (NIH). The government has certain rights in the invention.

### FIELD OF THE INVENTION

This invention relates generally to nanoparticle conjugates for treatment of cancer, as well as imaging methods and treatment methods using such nanoparticle conjugates.

### BACKGROUND

One of the current challenges in treating patients harboring epidermal growth factor receptor mutant (EGFRmt+) and platelet derived growth factor B (PDGFB)-driven malignant brain tumors is the limited CNS penetration of EGFR and PDGFR small molecule inhibitors (SMIs), such as gefitinib and dasatinib (das), respectfully, at standard daily dosing. The most common cancer to metastasize to the brain is non-small cell lung carcinoma (NSCLC), while glioblastoma multiforme (GBM) is the most common primary malignant brain tumor. As an attractive molecular candidate for targeted cancer therapy, epidermal growth factor receptor (EGFR) demonstrates activating mutations in 25% of metastatic NSCLCs and 40-50% of primary GBMs. These mutations are associated with a high response rate to EGFR tyrosine kinase inhibitors (TKIs), such as gefitinib. However, about one-third of patients develop central nervous system (CNS) metastases after responding to TKIs. This has been attributed to lower SMI concentrations in the brain or CSF, which are inadequate for killing EGFRmt+ tumor cells. Intermittent, high-dose therapy has been administered with only partial success to improve CNS responses in patients with EGFR-mutant (EGFRmt+) NSCLC. Currently, it remains challenging to achieve sufficient EGFR inhibitor concentrations in brain tissue to maximize treatment of primary malignant tumors or metastatic disease or in cerebrospinal fluid (CSF) to treat leptomeningeal metastases.

As another example, GBM requires aggressive local therapy and adjuvant chemotherapy to target widespread microscopic disease infiltration. However, such a treatment combination has conferred only short-term survival benefit, and alternative therapeutic strategies utilizing small molecule inhibitors (SMIs), such as dasatinib (BMS-354825), have been increasingly incorporated into treatment planning protocols. Dasatinib, a highly potent second-generation ATP-competitive inhibitor of multiple protein tyrosine kinases, including PDGFR and Src family kinases (SFKs), is known to reduce tumor cell survival, and proliferative and metastatic activity in vitro, however, most clinical trials that use this and other SMI's as monotherapies, have failed to demonstrate survival benefit in unselected malignant glioma patient populations.

There have been therapeutic attempts to regulate tumor microenvironments. For example, there have been recent therapeutic attempts to re-educate stromal cells within the tumor microenvironment to have anti-tumorigenic effects (see “Microenvironmental regulation of tumor progression and metastasis,” Daniela Quail and Johanna Joyce, Nature Medicine, Vol. 19, No. 11, November 2013). Previous work has used an inhibitor of the colony stimulating factor-1 (CSF-1) receptor (CSF-1R) to target tumor microenvironments in a mouse proneural GBM model, which significantly increased survival and regressed established tumors (see “CSF-1R inhibition alters macrophage polarization and blocks glioma progression,” Pyonteck et al., Nature Medicine, Vol. 19, no. 10, October 2013).

Furthermore, current strategies for genomically-defined metastatic disease to the brain are limited by variable and poor delivery through the blood-brain barrier, resulting in low tumor penetration at tolerable systemic doses.

These findings underscore the need to develop new drug delivery approaches and further elucidate key factors that might limit treatment response to EGFR inhibitors and other TKIs, such as bioavailability, penetration, serum protein binding, drug-specific properties, and non-specific tissue uptake. Moreover, there remains a need for a noninvasively quantifiable vehicle with enhanced drug delivery to primary and metastatic tumors (e.g., brain tumors), and to improve tumor delivery and therapeutic index of existing drugs in the treatment of primary and metastatic tumors.

### SUMMARY OF INVENTION

Described herein are nanoparticle conjugates that demonstrate enhanced penetration of tumor tissue (e.g., brain tumor tissue) and diffusion within the tumor interstitium, e.g., for treatment of cancer (e.g., primary and metastatic brain tumors). Further described are methods of targeting tumor-associated macrophages, microglia, and/or other cells in a tumor microenvironment using such nanoparticle conjugates. Moreover, diagnostic, therapeutic, and theranostic (diagnostic and therapeutic) platforms featuring such nanoparticle conjugates are described for treating targets in both the tumor and surrounding microenvironment, thereby enhancing efficacy of cancer treatment. Use of the nanoparticle conjugates described herein with other conventional therapies, including chemotherapy, radiotherapy, immunotherapy, and the like, is also envisaged.

Multi-targeted kinase inhibitors and combinations of single-targeted kinase inhibitors have been developed to overcome therapeutic resistance. Importantly, multimodality combinations of targeted agents, including particle-based probes designed to carry SMIs, chemotherapeutics, radiotherapeutic labels, and/or immunotherapies can enhance treatment efficacy and/or improve treatment planning of malignant brain tumors. Coupled with molecular imaging labels, these vehicles permit monitoring of drug delivery, accumulation, and retention, which may, in turn, lead to optimal therapeutic indices.

Moreover, use of radiolabels and/or fluorescent markers attached to (or incorporated in or on, or otherwise associated with) the nanoparticles provide quantitative assessment of particle uptake and monitoring of treatment response. In various embodiments, modular linkers are described for incorporating targeting ligands to develop a drug delivery system with controlled pharmacological properties. The described platforms determine the influence of targeting on nanoparticle penetration and accumulation, thereby establishing an adaptable platform for improved delivery of a range of tractable SMIs, for example, to primary and metastatic brain tumors.

In one aspect, the invention is directed to a method of treating cancer, the method comprising administering to a subject a pharmaceutical composition comprising a nanoparticle drug conjugate (NDC), the nanoparticle drug conjugate comprising: a nanoparticle with average diameter no greater than 20 nm; a linker moiety; and a drug moiety, wherein the drug moiety and the linker moiety form a cleavable linker-drug construct that is attached (e.g., covalently and/or non-covalently bound) to the nanoparticle, and wherein the NDC readily diffuses within tumor interstitium.

In certain embodiments, the cancer comprises a member selected from the group consisting of a malignant brain tumor, a metastatic brain tumor, non-small cell lung carcinoma (NSCLC) and a glioblastoma multiforme (GBM).

In certain embodiments, the method achieves sufficient drug moiety accumulation and/or (more uniform) distribution within tissue to treat a primary malignant tumor or metastatic disease. In certain embodiments, the method achieves sufficient drug moiety accumulation and/or (more uniform) distribution within cerebrospinal fluid so as to treat leptomeningeal metastases.

In certain embodiments, the nanoparticle has an average diameter from 3 to 8 nm.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme sensitive linker moiety.

In certain embodiments, the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

In certain embodiments, the nanoparticle drug conjugate comprises one or more targeting moieties (e.g., a targeting peptide) (e.g., a tumor-targeting moiety, e.g., an RGD-containing moiety, e.g., cRGDY, to target integrins (integrin receptors) and/or a microenvironment-targeting moiety e.g., αMSH to target melanocortin-1 receptors), (e.g., for delivering the drug moiety (e.g., small molecule inhibitors, SMIs) (e.g., to integrin- and/or melanocortin-1 (MC1)-expressing cells (e.g., tumor, macrophages))). In certain embodiments, the nanoparticle drug conjugate comprises from 1 to 20 discrete targeting moieties (e.g., of the same type or of different types).

In certain embodiments, the method comprises administering nanoparticle drug conjugates (e.g., multiple NDCs of the same or similar composition) with a first moiety for delivering and targeting the drug moiety to a tumor and NDCs with a second moiety for delivering and targeting the drug moiety to the microenvironment surrounding the tumor.

In certain embodiments, the first and second moieties may be on the same or different NDCs that are administered to the subject in one or more compositions.

In certain embodiments, the NDC comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety). In certain embodiments, the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the drug moiety comprises a SMI (e.g., CSF-1R, dasatinib) or a chemotherapeutic (e.g., sorafenib, paclitaxel, docetaxel, MEK162, etoposide, lapatinib, nilotinib, crizotinib, fulvestrant, vemurafenib, bexorotene, and/or camptotecin).

In certain embodiments, the nanoparticle drug conjugate comprises an immunomodulator and/or anti-inflammatory agent. In certain embodiments, the immunomodulator and/or anti-inflammatory agent comprises αMSH.

In certain embodiments, the method comprises administration (e.g., for immunotherapy) of an antibody or antibody fragment.

In certain embodiments, the composition comprises an antibody and/or an NDC with antibody fragment attached.

In certain embodiments, the method comprises administration of a NDC with antibody fragment attached, wherein the antibody fragment is a member selected from the set consisting of a recombinant antibody fragment (fAbs), a single chain variable fragment (scFv), and a single domain antibody (sdAb) fragment.

In certain embodiments, the antibody fragment is a single chain variable fragment (scFv). In certain embodiments, the antibody fragment is a single domain (sdAb) fragment.

In certain embodiments, the pharmaceutical composition comprises nanoparticles targeted to cancer cells such that the nanoparticles accumulate in concentrations sufficient to induce ferroptosis of the cancer cells.

In certain embodiments, the nanoparticle comprises silica (e.g., wherein the nanoparticle comprises a fluorescent compound, e.g., attached to and/or incorporated within the nanoparticle). In certain embodiments, the nanoparticle comprises a silica-based core and silica shell surrounding at least a portion of the core (e.g., wherein the nanoparticle comprises a fluorescent compound within the core).

In certain embodiments, the pharmaceutical composition comprises a carrier.

In another aspect, the invention is directed to a method of in vivo diagnosis and/or staging of cancer, wherein the in vivo diagnosis and/or staging comprises: delivering a pharmaceutical composition to the subject, wherein the pharmaceutical composition comprises a nanoparticle drug conjugate (NDC), the nanoparticle drug conjugate comprising: a nanoparticle with an average diameter no greater than 20 nm; a linker moiety; a drug moiety, wherein the drug moiety and the linker moiety form a cleavable linker-drug construct that is attached (e.g., covalently and/or non-covalently bound) to the nanoparticle, and wherein the NDC readily diffuses within tumor interstitium; and a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety); and detecting (e.g., via PET, x-ray, MRI, CT, etc.) the radioisotope in the subject.

In certain embodiments, the NDC comprises one or more targeting moieties (e.g., a targeting peptide) (e.g., a tumor-targeting moiety, e.g., an RGD-containing moiety, e.g., cRGDY targeting integrin receptors), and/or a microenvironment-targeting moiety, e.g., αMSH (targeting MC1-R), e.g., for delivering the drug moiety (e.g., the SMI).

In certain embodiments, the cancer comprises a member selected from the group consisting of a malignant brain tumor, a metastatic brain tumor, non-small cell lung carcinoma (NSCLC) and a glioblastoma multiforme (GBM).

In certain embodiments, the method achieves sufficient drug moiety accumulation and/or (more uniform) distribution within tissue to treat a primary malignant tumor or metastatic disease. In certain embodiments, the method achieves sufficient drug moiety accumulation and/or (more uniform) distribution within cerebrospinal fluid so as to treat leptomeningeal metastases.

In certain embodiments, the nanoparticle has an average diameter from 3 to 8 nm.

In certain embodiments, the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme sensitive linker moiety.

In certain embodiments, the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

In certain embodiments, the method comprises mapping a concentration of the radioisotope in the subject, e.g., in 2D or 3D, and, optionally, detecting fluorescence from a fluorescent compound (e.g., the fluorescent compound attached to and/or incorporated within the nanoparticle of the NDC).

In certain embodiments, the radioisotope detection/mapping step is part of a treatment of the cancer.

In certain embodiments, the method is a theranostic method.

In another aspect, the invention is directed to a pharmaceutical composition comprising a nanoparticle drug conjugate (NDC), the nanoparticle drug conjugate comprising: a nanoparticle with an average diameter no greater than 20 nm; a linker moiety; a drug, wherein the drug moiety and the linker moiety form a cleavable linker-drug construct that is attached (e.g., covalently and/or non-covalently bound) to the nanoparticle, and wherein the NDC readily diffuses within tumor interstitium; for use in a method of treating cancer, the method comprising administering to a subject a pharmaceutical composition comprising the nanoparticle drug conjugate.

In certain embodiments, the NDC comprises one or more targeting moieties (e.g., a targeting peptide) (e.g., a tumor-targeting moiety, e.g., an RGD-containing moiety, e.g., cRGDY, to target integrins (integrin receptors) and/or a microenvironment-targeting moiety e.g., αMSH to target melanocortin-1 receptors), e.g., for delivering the drug moiety (e.g., small molecule inhibitors, SMIs) (e.g., to integrin- and/or melanocortin-1 (MC1)-expressing cells (e.g., tumor, macrophages)).

In certain embodiments, the NDC comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety).

In certain embodiments, the cancer comprises a member selected from the group consisting of a malignant brain tumor, a metastatic brain tumor, non-small cell lung carcinoma (NSCLC) and a glioblastoma multiforme (GBM).

In certain embodiments, the method of treating cancer achieves sufficient drug moiety accumulation and/or (more uniform) distribution within tissue to treat a primary malignant tumor or metastatic disease. In certain embodiments, the method of treating cancer achieves sufficient drug moiety accumulation and/or (more uniform) distribution within cerebrospinal fluid so as to treat leptomeningeal metastases.

In certain embodiments, the nanoparticle has an average diameter from 3 to 8 nm

In certain embodiments, the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme cleavable linker.

In certain embodiments, the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

In certain embodiments, the pharmaceutical composition comprises a carrier.

In one aspect, the invention is directed to a pharmaceutical composition comprising a nanoparticle drug conjugate (NDC), the nanoparticle drug conjugate comprising: a nanoparticle with an average diameter no greater than 20 nm; a linker moiety; a drug moiety, wherein the NDC readily diffuses within tumor interstitium; for use in a method of in vivo diagnosis and/or staging of cancer, wherein the in vivo diagnosis and/or staging comprises: delivering the composition to the subject; and detecting (e.g., via PET, x-ray, MRI, CT, etc.) the radioisotope in the subject.

In certain embodiments, the NDC comprises one or more targeting moieties (e.g., a targeting peptide) (e.g., a tumor-targeting moiety, e.g., an RGD-containing moiety, e.g., cRGDY, to target integrins (integrin receptors) and/or a microenvironment-targeting moiety e.g., αMSH to target melanocortin-1 receptors), e.g., for delivering the drug moiety (e.g., small molecule inhibitors, SMIs) (e.g., to integrin- and/or melanocortin-1 (MC1)-expressing cells (e.g., tumor, macrophages)).

In certain embodiments, the NDC comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety).

In certain embodiments, the cancer comprises a member selected from the group consisting of a malignant brain tumor, a metastatic brain tumor, non-small cell lung carcinoma (NSCLC) and a glioblastoma multiforme (GBM).

In certain embodiments, the method achieves sufficient drug moiety accumulation and/or (more uniform) distribution within tissue to treat a primary malignant tumor or metastatic disease. In certain embodiments, the method achieves sufficient drug moiety accumulation and/or (more uniform) distribution within cerebrospinal fluid so as to treat leptomeningeal metastases.

In certain embodiments, the nanoparticle has an average diameter from 3 to 8 nm.

In certain embodiments, the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme sensitive linker.

In certain embodiments, the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

In certain embodiments, the method comprises mapping a concentration of the radioisotope in the subject, e.g., in 2D or 3D, and, optionally, detecting fluorescence from a fluorescent compound (e.g., the fluorescent compound attached to and/or incorporated within the nanoparticle of the NDC).

In certain embodiments, the radioisotope detection/mapping step is part of a treatment of the cancer.

In certain embodiments, the method is a theranostic method.

In certain embodiments, the pharmaceutical composition comprises a carrier.

In another aspect, the invention is directed to a pharmaceutical composition comprising a nanoparticle drug conjugate (NDC), the nanoparticle drug conjugate comprising: a nanoparticle with an average diameter no greater than 20 nm; a linker moiety; and a drug moiety, wherein the NDC readily diffuses within tumor interstitium.

In certain embodiments, the NDC comprises one or more targeting moieties (e.g., a targeting peptide) (e.g., a tumor-targeting moiety, e.g., an RGD-containing moiety, e.g., cRGDY, to target integrins (integrin receptors) and/or a microenvironment-targeting moiety e.g., αMSH to target melanocortin-1 receptors), e.g., for delivering the drug moiety (e.g., small molecule inhibitors, SMIs) (e.g., to integrin- and/or melanocortin-1 (MC1)-expressing cells (e.g., tumor, macrophages)).

In certain embodiments, the NDC comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety).

In certain embodiments, the tumor comprises a member selected from the group consisting of a malignant brain tumor, a metastatic brain tumor, non-small cell lung carcinoma (NSCLC), and a glioblastoma multiforme (GBM).

In certain embodiments, the NDC achieves sufficient drug moiety accumulation and/or (more uniform) distribution within tissue to treat a primary malignant tumor or metastatic disease. In certain embodiments, the NDC achieves sufficient drug moiety accumulation and/or (more uniform) distribution within cerebrospinal fluid so as to treat leptomeningeal metastases.

In certain embodiments, the nanoparticle has an average diameter from 3 to 8 nm.

In certain embodiments, the pharmaceutical composition comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme sensitive linker.

In certain embodiments, the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

In another aspect, the invention is directed to a method of manipulating (e.g., regulating, controlling) behavior of cells in a tumor microenvironment, the method comprising administering to a subject the pharmaceutical composition comprising a nanoparticle conjugate, the nanoparticle conjugate comprising: a nanoparticle with an average diameter no greater than 20 nm; a linker moiety (e.g., a cleavable linker, e.g., a biocleavable linker, e.g., a peptide, a hydrazone, a PEG, and/or a moiety comprising one or more amino acids (natural and/or non-natural amino acid)); and a modulator moiety, wherein the nanoparticle conjugate readily diffuses within tumor interstitium.

In certain embodiments, the nanoparticle conjugate comprises one or more targeting moieties (e.g., a targeting peptide) (e.g., a tumor-targeting moiety, e.g., an RGD-containing moiety, e.g., cRGDY, to target integrins (integrin receptors) and/or a microenvironment-targeting moiety e.g., αMSH to target melanocortin-1 receptors), e.g., for delivering the modulator moiety (e.g., to integrin- and/or melanocortin-1 (MC1)-expressing cells (e.g., tumor, macrophages)).

In certain embodiments, the nanoparticle conjugate comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety).

In certain embodiments, the tumor comprises a member selected from the group consisting of a malignant brain tumor, a metastatic brain tumor, non-small cell lung carcinoma (NSCLC) and a glioblastoma multiforme (GBM).

In certain embodiments, the nanoparticle has an average diameter from 3 to 8 nm.

In certain embodiments, the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

In certain embodiments, the linker moiety comprises a cleavable linker and/or a biocleavable linker. In certain embodiments, the linker moiety comprises a member selected from the group consisting of a peptide, a hydrazone, a PEG, and a moiety comprising one or more amino acids (natural and/or non-natural amino acid). In certain embodiments, the linker moiety comprises an enzyme sensitive linker.

In certain embodiments, the cells comprise a member selected from the group consisting of macrophages, tumor-associated macrophages and/or microglia (TAMs), dendritic cells, and T cells.

In certain embodiments, the tumor microenvironment is in vivo, in the treatment of cancer, brain cancer, malignant cancer, and/or malignant brain cancer.

In certain embodiments, the modulator moiety comprises an inhibitor of colony stimulating factor-1 (CSF-1R), for targeting TAMs, wherein the modulator moiety and the linker moiety form a cleavable linker-modulator construct that is attached (e.g., covalently and/or non-covalently bound) to the nanoparticle. In certain embodiments, the modular moiety comprises an immunomodulator (αMSH), wherein the modulator moiety and the linker moiety form a cleavable linker-modulator construct that is attached (e.g., covalently and/or non-covalently bound) to the nanoparticle.

It is contemplated that details and features described with respect to one aspect of the invention may be applied to another aspect of the invention.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7% 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous.

“Antibody”: As used herein, the term “antibody” refers to a polypeptide that includes canonical immunoglobulin sequence elements sufficient to confer specific binding to a particular target antigen. Intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH₁, CH₂, and the carboxy-terminal CH₃ (located at the base of the Y's stem). A short region, known as the “switch”, connects the heavy chain variable and constant regions. The “hinge” connects CH₂ and CH₃ domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH₂ domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. Affinity and/or other binding attributes of Fc regions for Fc receptors can be modulated through glycosylation or other modification. In certain embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In certain embodiments, an antibody is polyclonal; in certain embodiments, an antibody is monoclonal. In certain embodiments, an antibody has constant region sequences that are characteristic of mouse, rabbit, primate, or human antibodies. In certain embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In certain embodiments, an antibody may lack a covalent modification (e.g., attachment of a glycan) that it would have if produced naturally. In certain embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

“Antibodyfragment”: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in certain embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional single domain antibody fragment is in a range from about 5 kDa to about 25 kDa, e.g., from about 10 kDa to about 20 kDa, e.g., about 15 kDa; a functional single-chain fragment is from about 10 kDa to about 50 kDa, e.g., from about 20 kDa to about 45 kDa, e.g., from about 25 kDa to about 30 kDa; and a functional fab fragment is from about 40 kDa to about 80 kDa, e.g., from about 50 kDa to about 70 kDa, e.g., about 60 kDa.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In certain embodiments, associated moieties are covalently linked to one another. In certain embodiments, associated entities are non-covalently linked. In certain embodiments, associated entities are linked to one another by specific non-covalent interactions (e.g., by interactions between interacting ligands that discriminate between their interaction partner and other entities present in the context of use, such as, for example, streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In certain embodiments, biodegradable materials are enzymatically broken down. Alternatively or additionally, in certain embodiments, biodegradable materials are broken down by hydrolysis. In certain embodiments, biodegradable polymeric materials break down into their component polymers. In certain embodiments, breakdown of biodegradable materials (including, for example, biodegradable polymeric materials) includes hydrolysis of ester bonds. In certain embodiments, breakdown of materials (including, for example, biodegradable polymeric materials) includes cleavage of urethane linkages.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Cancer”: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In certain embodiments, a cancer may be characterized by one or more tumors. In certain embodiments, the cancer is a malignant brain tumor, a metastatic brain tumor, non-small cell lung carcinoma (NSCLC) or a glioblastoma multiforme (GBM). Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, vulva, malignant brain tumors, metastatic brain tumors, non-small cell lung carcinoma (NSCLC), and/or a glioblastoma multiforme (GBM).

“Chemotherapeutic Agent” or “Drug”: As used herein, the term “chemotherapeutic agent” or “drug” (e.g., anti-cancer drug) has its art-understood meaning referring to one or more pro-apoptotic, cytostatic and/or cytotoxic agents, for example, specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g., microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, all-trans retinoic acid, an Auiristatin, Azacitidine, Azathioprine, Bleomycin, Bortezomib, Carboplatin, Capecitabine, Cisplatin, Chlorambucil, Cyclophosphamide, curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be or comprise one or more of famesyl-thiosalicylic acid (FTS), 4-(4-Chloro-2-methylphenoxy)-N-hydroxybutanamide (CMH), estradiol (E2), tetramethoxystilbene (TMS), S-tocatrienol, salinomycin, or curcumin.

“Imaging agent”: As used herein, “imaging agent” refers to any element, molecule, functional group, compound, fragments thereof or moiety that facilitates detection of an agent (e.g., a polysaccharide nanoparticle) to which it is joined. Examples of imaging agents include, but are not limited to: various ligands, radionuclides (e.g., ³H, ¹⁴C, ¹⁸F, ¹⁹F, ³²P, ³⁵S, ¹³⁵I, ¹²⁵I, ¹²⁴I, ¹²³I, ¹³¹I, ⁶⁴Cu, ⁶⁸Ga, ¹⁸⁷Re, ¹¹¹In, ⁹⁰Y, ^{99m}Tc, ¹⁷⁷Lu, ⁸⁹Zr) fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available. The radionuclides may be attached via click chemistry, for example.

“Nanoparticle”: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In certain embodiments, very small nanoparticles are used, for example, nanoparticles having average diameter no greater than 20 nm, e.g., no greater than 15 nm, e.g., no greater than 10 nm, e.g., from 3 nm to 8 nm) (e.g., with a size distribution such that at least 85 wt. % of the nanoparticles (e.g., at least 85 wt. %, at least 90 wt. %, at least 95 wt. %, at least 98 wt. %, or at least 99 wt. %) is no greater than 20 nm, e.g., no greater than 15 nm, e.g., no greater than 10 nm, e.g., from 3 nm to 8 nm). In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

“Peptide” or “Polypeptide”: The term “peptide” or “polypeptide” refers to a string of at least two (e.g., at least three) amino acids linked together by peptide bonds. In certain embodiments, a polypeptide comprises naturally-occurring amino acids; alternatively or additionally, in certain embodiments, a polypeptide comprises one or more non-natural amino acids (i.e., compounds that do not occur in nature but that can be incorporated into a polypeptide chain; see, for example, http://www.cco.caltech.edu/^{˜}dadgrp/Unnatstruct.gif, which displays structures of non-natural amino acids that have been successfully incorporated into functional ion channels) and/or amino acid analogs as are known in the art may alternatively be employed). In certain embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Pharmaceutical composition”: As used herein, the term “pharmaceutical composition” refers to an active agent, formulated together with one or more pharmaceutically acceptable carriers. In certain embodiments, active agent is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Radiolabel” or “Radioisotope”: As used herein, “radiolabel” or “radioisotope” refers to a moiety comprising a radioactive isotope of at least one element. Exemplary suitable radiolabels include but are not limited to those described herein. In certain embodiments, a radiolabel is one used in positron emission tomography (PET). In certain embodiments, a radiolabel is one used in single-photon emission computed tomography (SPECT). In certain embodiments, radioisotopes comprise ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, and ¹⁹²Ir.

“Subject”: As used herein, the term “subject” includes humans and mammals (e.g., mice, rats, pigs, cats, dogs, and horses). In many embodiments, subjects are mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent”, “Drug”, “Pharmaceutical Composition”: As used herein, the terms “therapeutic agent”, “drug”, and “pharmaceutical composition” refer to any agent that has a therapeutic effect and/or elicits a desired biological and/or pharmacological effect, when administered to a subject.

“Therapeutically effective amount”: as used herein, “therapeutically effective amount” refers to an amount that produces the desired effect for which it is administered. In certain embodiments, the term refers to an amount that is sufficient, when administered to a population suffering from or susceptible to a disease, disorder, and/or condition in accordance with a therapeutic dosing regimen, to treat the disease, disorder, and/or condition. In certain embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In certain embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in certain embodiments, a therapeutically effective amount of a particular agent or therapy may be formulated and/or administered in a single dose. In certain embodiments, a therapeutically effective agent may be formulated and/or administered in a plurality of doses, for example, as part of a dosing regimen.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain embodiments, treatment may be of a subject who has been diagnosed as suffering from the relevant disease, disorder, and/or condition. In certain embodiments, treatment may be of a subject known to have one or more susceptibility factors that are statistically correlated with increased risk of development of the relevant disease, disorder, and/or condition.

Drawings are presented herein for illustration purposes, not for limitation.

### DESCRIPTION OF DRAWINGS

The foregoing and other objects, aspects, features, and advantages of the present disclosure will become more apparent and better understood by referring to the following description taken in conduction with the accompanying drawings, in which:

FIGS. 1A-1B are an example cleavable linker-drug construct attached to an ultrasmall particle (e.g., wherein average particle diameter ≤20 nm, ≤15 nm, or ≤10 nm), is illustrated. The figures demonstrate protease mediated drug release in cells by detachment of the drug moiety at the enzyme cleavage site following arrival of the nanoparticle drug conjugate at the targeted location. The figures depict ultrasmall silica nanoparticles for delivery of small molecule inhibitors, in accordance with illustrative embodiments of the invention. For example, the nanoparticles deliver small molecule inhibitors (SMIs) to primary and metastatic brain tumors with improved therapeutic index.

FIGS. 2-6 are images from experiments with a platelet-derived growth factor B (PDGFB)-driven mouse model of high grade glioma.

FIGS. 7-9 are images from experiments demonstrating integrin expression and particle uptake in a RCAS-PDGFB glioma model.

FIG. 10 is a chart illustrating use of the RCAS-PDGFB mouse glioma model to study C′-dot distribution via concurrent intravital staining. The figures depict that mice were given in vivo injections of RGD-targeted C′ dots and sacrificed at 3 or 96 hours. 70 kDa FITC-labeled Dextran served as a surrogate marker for blood brain barrier (BBB) breakdown. Hoeschst staining was used to demonstrate nuclear localization.

FIG. 11 shows images from an ex vivo study of cRGD-Cy5-C′-dot distribution in RCAS tumor-bearing mice.

FIG. 12 shows images from an ex vivo study of ¹²⁴I-RGD-Cy5-C′-dot distribution in RCAS tumor-bearing mice.

FIGS. 13A-13B are images from in vivo baseline studies, using the base particle probe (i.e., FDA-IND approved cRGDY-PEG-C′ dots) in conjunction with time-dependent intravital staining methods to provide initial assessments of intratumoral penetration and particle distribution kinetics as a function of blood-brain barrier permeability, integrin-targeting (vs non-integrin targeting using cRADY-PEG-C′ dots).

FIG. 14 are images obtained after 96 hours show the nanoparticle with RGD exhibited greater diffused in the tumor than the nanoparticle with RAD. FIG. 14 also shows an image of 70 kDa FITC-labeled Dextran 3 hours after administration, as a reference tracer of similar size to the nanoparticle conjugates, which is suggestive of intracellular localization of Cy5-C′ dots at least as early as three hours post-treatment.

FIGS. 15A-15F are triple fluorescence labeling images of FITC-Dextran as a reference tracer of similar size to the nanoparticle conjugates of FIGS. 13A-13B. As explained above, the data is suggestive of intracellular localization of Cy5-C′ dots at least as early as three hours post-treatment.

FIG. 16 are MRI-PET and histological images of ¹²⁴I-cRGDY-PEG-C′ dots in brain tumors.

FIGS. 17 and 18 are western blot images, fluorescence images, and microscope images that demonstrate that gefitinib-C′-dots retain potency in H1650 cells comparable to free drug (or improved). RGD-C′-dots are internalized in H1650 cell lysosomes, and describes optimization of delivery and release of small molecule inhibitors (SMI) from nanoparticle drug conjugates (NDCs) (e.g., Yoo et al 2015, Bioorg Med Chem). For example, CNS drug levels limit clinical use of SMIs even for sensitive brain tumors. Gefitnib can be used as a tool to assess nanoparticle-drug potency and kinetics.

FIG. 19 are images of H1650 flank xenografts treated with Gef-NDC. The images show particle-specific fluorescence and achieve pEGFR inhibition in a time-dependent fashion—this is relevant to the determination of drug delivery and potency of NDCs in NSCLC tumor-bearing mice.

FIGS. 20 and 21 show experimental results relevant to the characterization of gefitinib and gef-NDC response in patient-derived EGFR L858R NSCLC line (ECLC26).

FIG. 20 shows viability of ECLC26 vs. gefitinib (from 1 nM to 1 μM) for 72 hours.

FIG. 21 shows phosphor-EGFR inhibition in ECLC26 by gefitinib and Type II gef-NDC.

FIG. 22 shows an illustrative linker chemical structure relevant to the development and testing of dasatinib NDC for investigation in the RCAS-PDGF glioma model.

FIGS. 23A-23F are images that demonstrate “pulsatile” high-dose erlotinib improves CNS penetration for NSCLC metastases. Response of CNS metastases to pulsatile erlotinib in 3 patients are shown. Grommes et al., Neuro Oncol., 2011 Dec. 13(12): 1364-9. While a response is apparent, the response is unpredictable, even at high dose.

FIGS. 23A and 23B are contrast (gadolinium)-enhanced axial T1 MRI sequences in patient #3 with leptomeningeal metastases (arrows) before (FIG. 23A) and after (FIG. 23B) 6 months of therapy.

FIGS. 23C and 23D are images taken in Patient #6 with coexistent brain (large arrow) and leptomeningeal metastases (not shown) before (FIG. 23C) and after (FIG. 23D) 5 months of therapy.

FIGS. 23E and 23F are images in Patient #8 with coexistent brain (arrow heads) and leptomeningeal metastases (not shown) before (FIG. 23E) and after (FIG. 23F) 2 months of therapy.

FIG. 24 are MRI-PET and histological imaging of ¹²⁴I-cRGDY-PEG-C′ dots in brain tumors.

FIG. 25 are images from an ex vivo study of cRGD-C′-dot distribution in mouse glioma. Triple fluorescence labeling of RAD-nanoparticle (NP) at 3 hours demonstrates that there is no difference between Cy5 signal and FITC signal, thereby suggesting that the non-targeted particle does not significantly diffuse past regions of blood brain barrier breakdown at this time point.

FIG. 26 are images and quantitative data of RGD vs. RAD compared at 96 hours.

FIGS. 27 and 28 are images and data depicting distribution analysis by pixel correlation. High-powered imaging of focal regions of tumor treated with targeted and non-targeted nanoparticle. RCAS-tva tumor bearing mice were treated with either radiolabeled RGD-targeted nanoparticle or RAD-nanoparticle and sacrificed at 96 hours post-treatment with injection of FITC-Dextran 3 hours prior to sacrifice. Frozen sections were analyzed for fluorescent signal using high-powered imaging of representative regions. The data demonstrates closely overlapping regions of RAD-nanoparticle signal with regions of BBB breakdown as marked by FITC, compared to a more diffuse pattern of Cy5 signal beyond FITC hotspots in RGD-nanoparticle treated tumors. Each animal has 4 to 5 regions averaged per section (N=4 mice).

FIG. 29 is an image of a western blot indicating that dasatinib-NDC achieves PDGFR inhibition in a dose-dependent manner at levels similar to free drug. TS543 cells (neurosphere tumor line) harboring a PDGFRA Δ8, 9, constitutively activating mutation were treated with the indicated drugs for 4 hours followed by PDGF-BB 20 ng/ml for 10 minutes.

FIG. 30 are images of dasatinib-NDC distribution in tumor at 3 and 96 hours post-treatment.

FIG. 31 are H&E and fluorescence images of comparable distribution of fluorescent signal in targeted and non-targeted nanoparticle-drug conjugate compared to targeted and non-targeted particle alone.

FIG. 32 are H&E images showing that gefitinib-NDC achieves p-EGFR target inhibition at 18 hours post-treatment. ECLC 26 tumor-bearing mice were treated with either Gefitinib-NDC, Gefitinb P.O. (150 mg/kg), or oral saline vehicle and sacrificed at 18 hours post-treatment. Tumors were embedded in paraffin and sectioned and stained with p-EGFR and H&E.

FIG. 33 shows data from a multi-dose treatment of ECLC26 flank tumor bearing mice that results in robust tumor control.

FIG. 34 are western blow images that show ECLC26 growth post treatments using the NDCs provided herein.

FIG. 35 shows histological images indicating that 45 μM RGD-Das-NDC effectively inhibited target in primary gliomas compared to untreated controls after 24 hours. Brain tissue was harvested at 24 hours after i.v. injection of the nanoparticle drug conjugates, and was stained for the expression of phosphor s6 ribosomal protein. Growth factors and mitogens induce the activation of p70 S6 kinase and the subsequent phosphorylation of the S6 ribosomal protein. Phosphorylation of S ribosomal protein correlates with an increase in translation of mRNA transcripts that contain an oligopyrimidine tract in their 5′ untranslated regions. These particular mRNA transcripts (5′TOP) encode proteins involved in cell cycle progression, as well as ribosomal proteins and elongation factors necessary for translation. S6 ribosomal protein phosphorylation sites include several residues (Ser235, Ser236, Ser240, and Ser244) located within a small, carboxy-terminal region of the S6 protein.

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Various embodiments described herein utilize ultrasmall, sub-10 nm FDA-IND approved fluorescent organo-silica particles (C dots), and/or ultrasmall poly(ethylene glycol)-coated (PEGylated) near-infrared (NIR) fluorescent silica nanoparticle, referred to as C′ dots. For example, in certain embodiments, the C dots or C′ dots are surface-adapted with one or more PET radiolabels and one or more targeting ligands (e.g., the integrin-targeting peptide cyclo-(Arg-Gly-Asp-Tyr) (cRGDY)). Detail on C dots are described in U.S. Pat. No. 8,298,677 B2 “Fluorescent silica-based nanoparticles”, U.S. Publication No. 2013/0039848 A1 “Fluorescent silica-based nanoparticles”, U.S. Publication No. US 2014/0248210 A1 “Multimodal silica-based nanoparticles”, U.S. Publication No. US 2015/0366995 A1 “Mesoporous oxide nanoparticles and methods of making and using the same” and U.S. Publication No. US 2016/0018404 A1 “Multilayer fluorescent nanoparticles and methods of making and using same”, the contents of which are incorporated herein by reference in their entireties.

C dots (or C′ dots) provide a unique platform for drug delivery due to their physical properties as well as demonstrated human in vivo characteristics. These particles are ultrasmall and benefit from EPR effects in tumor microenvironments, while retaining desired clearance and pharmacokinetic properties. To this end, described herein is a nanoparticle drug delivery system in which, in certain embodiments, drug constructs are covalently attached to C dots (or other nanoparticles). C dot-based (or C′ dot-based) NDCs for drug delivery provide good biostability, minimize premature drug release, and exhibit controlled release of the bioactive compound. In certain embodiments, peptide-based linkers are used for NDC applications. These linkers, in the context of antibodies and polymers, are stable both in vitro and in vivo, with highly predictable release kinetics that rely on enzyme catalyzed hydrolysis by lysosomal proteases. For example, cathepsin B, a highly expressed protease in lysosomes, can be utilized to facilitate drug release from macromolecules. By incorporating a short, protease sensitive peptide between the macromolecular backbone and the drug molecule, controlled release of the drug can be obtained in the presence of the enzyme.

In certain embodiments, the NDCs are ultrasmall (e.g., with average diameter from about 5 nm to about 10 nm, (e.g., about 6 nm)) and utilize enzyme sensitive linkers, for example, where drug release is catalyzed by proteases. In one example, gefitinib, an important epidermal growth factor receptor mutant (EGFRmt+)-tyrosine kinase inhibitor (TKI) cancer drug, was modified and incorporated onto the particles. The resulting NDCs exhibited excellent in vitro stability, solubility, and proved to be active in EGFRmt+-expressing NSCLC cells.

In certain embodiments, the NDCs comprise one or more targeting moieties, for example, to target a particular tissue type (e.g., a particular tumor). NDCs with target moieties enhance internalization of drugs in tumor cells (e.g., targeting ligands bind to receptors on tumor cells, and/or deliver drugs into tumor cells (e.g., by increased permeability)). For example, to create a particle therapeutic with an additional targeting moiety (e.g., cRGD), silica nanoparticles are added to a mixture of cRGDY-PEG conjugates and maleimide bifunctionalized PEGs. The maleimide bifunctionalized PEGs support the additional attachment of drug-linker conjugates to create a theranostic product.

In some embodiments, ultrasmall particles may be associated with PET labels and/or optical probes. Nanoparticles may be observed in vivo (e.g., via PET) to evaluate drug accumulation in a target site. For example, nanoparticles with PET labels (e.g., without drug substances) may be administered first. Then, by analyzing the in vivo PET images of the nanoparticles, drug (e.g., conjugated with nanoparticles) concentration and accumulation rate in the tumor may be estimated. The dose may be determined based on the obtained estimation to provide personalized medicine (e.g., tumor size rather than the patient's body weight). In some embodiments, a radiolabeled drug may be traced in vivo. A highly concentrated chemotherapy drug is potentially dangerous if it is not targeted. In some embodiments, nanoparticles with optical probes (e.g., fluorophore) may be used for intraoperative imaging (e.g., where surface of tissue/tumor is exposed) and/or biopsies of tumors.

The therapeutic agent and nanoparticle can be radiolabeled or optically labelled separately, allowing independent monitoring of the therapeutic agent and the nanoparticle. In one embodiment, radiofluorinated (i.e., ¹⁸F) dasatinib is coupled with PEG-3400 moieties attached to the nanoparticle via NHS ester linkages. Radiofluorine is crucial for being able to independently monitor time-dependent changes in the distribution and release of the drug from the radioiodinated C24I) fluorescent (Cy5) nanoparticle. In this way, the pro drug (dasatinib) and nanoparticle can be monitored. This permits optimization of the prodrug design compared with methods in the prior art where no dual-labeling approach is used. In another embodiment, radiotherapeutic iodine molecules (e.g., ¹³¹I), or other therapeutic gamma or alpha emitters, are conjugated with PEG via a maleimide functional group, where the therapeutic agent may not dissociate from the PEG in vivo.

In various embodiments, NDCs are drug compounds covalently attached to C dot nanoparticles (or other nanoparticles (e.g., C′ dots)) through a molecular linker. In certain embodiments, linkers incorporate peptide (e.g., dipeptide) sequences sensitive to trypsin (control enzyme) and/or cathepsin B, which is an enzyme found predominantly in the lysosomes of cells. In certain embodiments, a class of linker chemistries that incorporates an amide bond between the linker and drug. In certain embodiments, a class of linker chemistries that utilize a degradable moiety between the linker and drug. In some embodiments, the linkers are designed to release the drug from the nanoparticle (e.g., C dot, e.g., C′ dot) under particular conditions, for example, proteolytic hydrolysis.

Example drugs that can be used include RTK inhibitors, such as dasatinib and gefitinib, can target either platelet-derived growth factor receptor (PDGFR) or EGFRmt+ expressed by primary tumor cells of human or murine origin (e.g., genetically engineered mouse models of high-grade glioma, neurospheres from human patient brain tumor explants) and/or tumor cell lines of non-neural origin. Dasatinib and gefitinib analogs can be synthesized to enable covalent attachment to several linkers without perturbing the underlying chemical structure defining the active binding site.

Synthetic approaches were validated and the desired linker-drug constructs and NDCs were obtained as described in International Application No. PCT/US2015/032565 (published as WO 2015/183882 on Dec. 3, 2015), the contents of which are hereby incorporated by reference in its entirety.

C dots or C′ dots can also serve as highly specific and potent multi-therapeutic targeted particle probes to combine antibody fragments with therapeutic radiolabels (e.g., ¹⁷⁷Lu, ²²⁵Ac, ⁹⁰Y, ⁸⁹Zr) on a single platform. Alternatively, C dot or C′ dot coupling of targeting peptides, such as alphaMSH, known to be immunomodulatory and anti-inflammatory in nature, can also be combined with C dot or C′ dot radiotherapeutic (and/or other particle-based) platforms to achieve enhanced efficacy. In certain embodiments, the concentration of the radioisotope and/or antibody fragment is higher in therapeutic applications compared to diagnostic applications.

Molecular therapeutics (e.g., antibodies) can modulate the immune system toward antitumor activity by manipulating immune checkpoints (e.g., the monoclonal antibody ipilimumab inhibits CTLA4, a negative regulatory molecule that inhibits function of the immune system). The rationale is to trigger preexisting, but dormant, antitumor immune responses. Other molecules and pathways have acted as immune switches. PD-1, another negative regulatory receptor expressed on T cells, has also been targeted. Switching a single immune checkpoint may not be sufficient to induce an antitumor response, explaining some of the failures of targeting single immune regulatory checkpoints like PD-1 or CTLA4. However, without wishing to be bound to any theory, treatment can be bolstered by the addition of RT, which is thought, in some cases, to have immunomodulatory properties. In these cases, tumors outside of RT treatment fields have been found to shrink as a result of a putative systemic inflammatory or immune response provoked by RT, highlighting the potential for radiation to spark a systemic antitumor immune response. Augmenting immune activity may also potentiate the local effects of RT.

By increasing the concentration alone of these immunoconjugates, disease can be treated. A therapeutic radiolabel can also be added to further treat disease. In certain embodiments, the immunoconjugate act as a therapeutic at high concentrations, and without a therapeutic radiolabel. In certain embodiments, the radiolabel is attached to the same nanoparticle in an all-in-one multi-therapeutic platform. Alternatively, therapeutic radioisotopes can be administered independently. More detail is provided in International Application No. PCT/US16/26434 (published as WO 2016/164578 on Oct. 13, 2016), the contents of which are hereby incorporated by reference in its entirety.

In contrast to other multimodal platforms, immunoconjugates can comprise different moieties that are attached to the nanoparticle itself. For example, in certain embodiments, a radioisotope is attached to the nanoparticle and an antibody fragment is attached to the nanoparticle—that is, in these embodiments, the radiolabel is not attached to the antibody fragment itself. As another example, immunoconjugates can comprise a targeting ligand attached to the nanoparticle, a radioisotope attached to the nanoparticle, and an antibody fragment attached to the nanoparticle. The stoichiometric ratios of different moieties attached to the C dot can affect the biodistribution of the nanoparticle immunoconjugate.

In certain embodiments, the nanoparticle comprises silica, polymer (e.g., poly(lactic-co-glycolic acid) (PLGA)), biologics (e.g., protein carriers), and/or metal (e.g., gold, iron). In certain embodiments, the nanoparticle is a “C dot” as described in U.S. Publication No. 2013/0039848 A1 by Bradbury et al., which is hereby incorporated by reference.

In certain embodiments, the nanoparticle is spherical. In certain embodiments, the nanoparticle is non-spherical. In certain embodiments, the nanoparticle is or comprises a material selected from the group consisting of metal/semi-metal/non-metals, metal/semi-metal/non-metal-oxides, -sulfides, -carbides, -nitrides, liposomes, semiconductors, and/or combinations thereof. In certain embodiments, the metal is selected from the group consisting of gold, silver, copper, and/or combinations thereof.

The nanoparticle may comprise metal/semi-metal/non-metal oxides including silica (SiO₂), titania (TiO₂), alumina (Al₂O₃), zirconia (ZᵣO2), germania (GeO₂), tantalum pentoxide (Ta₂O₅), NbO₂, etc., and/or non-oxides including metal/semi-metal/non-metal borides, carbides, sulfide and nitrides, such as titanium and its combinations (Ti, TiB₂, TiC, TiN, etc.).

The nanoparticle may comprise one or more polymers, e.g., one or more polymers that have been approved for use in humans by the U.S. Food and Drug Administration (FDA) under 21 C.F.R. § 177.2600, including, but not limited to, polyesters (e.g., polylactic acid, poly(lactic-co-glycolic acid), polycaprolactone, polyvalerolactone, poly(1,3-dioxan-2-one)); polyanhydrides (e.g., poly(sebacic anhydride)); polyethers (e.g., polyethylene glycol); polyurethanes; polymethacrylates; polyacrylates; polycyanoacrylates; copolymers of PEG and poly(ethylene oxide) (PEO).

The nanoparticle may comprise one or more degradable polymers, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

In certain embodiments, a nanoparticle can have or be modified to have one or more functional groups. Such functional groups (within or on the surface of a nanoparticle) can be used for association with any agents (e.g., detectable entities, targeting entities, therapeutic entities, or PEG). In addition to changing the surface charge by introducing or modifying surface functionality, the introduction of different functional groups allows the conjugation of linkers (e.g., (cleavable or (bio-) degradable) polymers such as, but not limited to, polyethylene glycol, polypropylene glycol, PLGA, etc.), targeting/homing agents, and/or combinations thereof.

In certain embodiments, the nanoparticle comprises one or more targeting ligands (e.g., attached thereto), such as, but not limited to, small molecules (e.g., folates, dyes, etc.), aptamers (e.g., A10, AS1411), polysaccharides, small biomolecules (e.g., folic acid, galactose, bisphosphonate, biotin), oligonucleotides, and/or proteins (e.g., (poly) peptides (e.g., αMSH, RGD, octreotide, AP peptide, epidermal growth factor, chlorotoxin, transferrin, etc.), antibodies, antibody fragments, proteins, etc.). In certain embodiments, the nanoparticle comprises one or more contrast/imaging agents (e.g., fluorescent dyes, (chelated) radioisotopes (SPECT, PET), MR-active agents, CT-agents), and/or therapeutic agents (e.g., small molecule drugs, therapeutic (poly)peptides, therapeutic antibodies, (chelated) radioisotopes, etc.).

In certain embodiments, PET (Positron Emission Tomography) tracers are used as imaging agents. In certain embodiments, PET tracers comprise ⁸⁹Zr, ⁶⁴Cu, [¹⁸F] fluorodeoxyglucose. In certain embodiments, the nanoparticle includes these and/or other radiolabels.

In certain embodiments, the nanoparticle comprises one or more fluorophores. Fluorophores comprise fluorochromes, fluorochrome quencher molecules, any organic or inorganic dyes, metal chelates, or any fluorescent enzyme substrates, including protease activatable enzyme substrates. In certain embodiments, fluorophores comprise long chain carbophilic cyanines. In other embodiments, fluorophores comprise DiI, DiR, DiD, and the like. Fluorochromes comprise far red, and near infrared fluorochromes (NIRF). Fluorochromes include but are not limited to a carbocyanine and indocyanine fluorochromes. In certain embodiments, imaging agents comprise commercially available fluorochromes including, but not limited to Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); and ADS780WS, ADS830WS, and ADS832WS (American Dye Source) and Kodak X-SIGHT 650, Kodak X-SIGHT 691, Kodak X-SIGHT 751 (Carestream Health).

In certain embodiments, the nanoparticle comprises (e.g., has attached) one or more targeting ligands, e.g., for targeting cancer tissue/cells of interest.

In certain embodiments, the nanoparticles comprise from 1 to 20 discrete targeting moieties (e.g., of the same type or different types), wherein the targeting moieties bind to receptors on tumor cells (e.g., wherein the nanoparticles have an average diameter no greater than 15 nm, e.g., no greater than 10 nm, e.g., from about 5 nm to about 7 nm, e.g., about 6 nm). In certain embodiments, the 1 to 20 targeting moieties comprises alpha-melanocyte-stimulating hormone (αMSH). In certain embodiments, the nanoparticles comprise a targeting moiety (e.g., αMSH).

In certain embodiments, the compositions and methods described herein induce cell death via ferroptosis by nanoparticle ingestion. Moreover, the present disclosure describes the administration of high concentrations of ultrasmall (e.g., having a diameter no greater than 20 nm, e.g., no greater than 15 nm, e.g., no greater than 10 nm) nanoparticles at multiple times over the course of treatment in combination with a nutrient-depleted environment, thereby modulating cellular metabolic pathways to induce cell death by the mechanism ferroptosis. Ferroptosis involves iron, reactive oxygen species, and a synchronous mode of cell death execution. More detail is provided in International Application No. PCT/US16/34351 (published as WO 2016/196201 on Dec. 8, 2016), the contents of which are hereby incorporated by reference in its entirety.

Cancers that may be treated include, for example, prostate cancer, breast cancer, testicular cancer, cervical cancer, lung cancer, colon cancer, bone cancer, glioma, glioblastoma, multiple myeloma, sarcoma, small cell carcinoma, melanoma, renal cancer, liver cancer, head and neck cancer, esophageal cancer, thyroid cancer, lymphoma, pancreatic (e.g., BxPC3), lung (e.g., H1650), and/or leukemia.

In certain embodiments, the nanoparticle comprises a therapeutic agent, e.g., a drug moiety (e.g., a chemotherapy drug) and/or a therapeutic radioisotope. As used herein, “therapeutic agent” refers to any agent that has a therapeutic effect and/or elicits a desired biological and/or pharmacological effect, when administered to a subject.

In certain embodiments, e.g., where combinational therapy is used, an embodiment therapeutic method includes administration of the nanoparticle and administration of one or more drugs (e.g., either separately, or conjugated to the nanoparticle), e.g., one or more chemotherapy drugs, such as sorafenib, paclitaxel, docetaxel, MEK162, etoposide, lapatinib, nilotinib, crizotinib, fulvestrant, vemurafenib, bexorotene, and/or camptotecin.

The surface chemistry, uniformity of coating (where there is a coating), surface charge, composition, concentration, frequency of administration, shape, and/or size of the nanoparticle can be adjusted to produce a desired therapeutic effect.

Described herein are nanoparticle conjugates that demonstrate enhanced penetration of tumor tissue (e.g., brain tumor tissue) and diffusion within the tumor interstitium, e.g., for treatment of cancer. Further described are methods of targeting tumor-associated macrophages, microglia, and/or other cells in a tumor microenvironment using such nanoparticle conjugates. Moreover, diagnostic, therapeutic, and theranostic (diagnostic and therapeutic) platforms featuring such nanoparticle conjugates are described for treating targets in both the tumor and surrounding microenvironment, thereby enhancing efficacy of cancer treatment. Use of the nanoparticle conjugates described herein with other conventional therapies, including chemotherapy, radiotherapy, immunotherapy, and the like, is also envisaged.

Multi-targeted kinase inhibitors and combinations of single-targeted kinase inhibitors have been developed to overcome therapeutic resistance. Importantly, multimodality combinations of targeted agents, including particle-based probes designed to carry SMIs, chemotherapeutics, radiotherapeutic labels, and/or immunotherapies can enhance treatment efficacy and/or improve treatment planning of malignant brain tumors. Coupled with molecular imaging labels, these vehicles permit monitoring of drug delivery, accumulation, and retention, which may, in turn, lead to optimal therapeutic indices.

One such clinically translated ultrasmall nanoparticle (e.g., a nanoparticle having a diameter no greater than 20 nm, e.g., no greater than 15 nm, e.g., no greater than 10 nm) platform, C′ dots, is useful for this purpose. This nanoparticle has been developed as a tumor-targeting dual-modality (PET-optical) drug delivery vehicle. Their favorable kinetic, internalizing, and enhanced tumor retention properties, along with their ability to readily diffuse within the tumor interstitium, have suggested that systemic delivery of these particles to the CNS and their more widespread distribution within the extracellular matrix, may be adequate to achieve therapeutic concentrations and improve targeted treatment response. New nanoparticle drug conjugates (NDCs) have been synthesized and characterized for the controlled delivery of prototype EGFR (gefitinib, gef) and PDGFR (dasatinib, das) SMIs to EGFRmt+ and PDGFB-driven tumor models, respectively. SMIs were attached to the particle surface using several different linker chemistries; loading and release profiles assessed in serum-supplemented media.

In certain embodiments, the nanoparticles have an average diameter no greater than 15 nm. In certain embodiments, the nanoparticles have an average diameter no greater than 10 nm. In certain embodiments, the nanoparticles have an average diameter from about 5 nm to about 7 nm (e.g., about 6 nm).

### EXAMPLES

The present Examples provide for a two-pronged approach to demonstrate feasibility of the nanoparticle platform described herein for treating tumors in subjects, particularly metastatic brain tumors. The first prong of the two-pronged approach uses a primary glioma model to understand behavior and distribution of a nanoparticle in a tumor (e.g., if a drug is on the particle, does the particle effectively treat the tumor compared to free drugs). The second prong of the two-pronged approach uses nanoparticle drug conjugates (NDCs) to treat and/or regulate tumor microenvironment to change phenotype of macrophages (e.g., in metastatic brain tumor). As described in detail herein, xenographs were created to establish the efficacy of the provided compositions in vivo and established the described compositions for treatment in the brain. The Examples demonstrate that tumor targeting is achieved with and/or without the attachment of a targeting moiety to the nanoparticle compositions. There is evidence the use of a targeting moiety improves transport and/or concentration of the nanoparticles to/into the tissue/tumor of interest.

### Example 1: Distribution, Efficacy, and Optimized Dosing of C′-Dots in Brain Tumors

The present Example provides for (1) determining the intratumoral and intracellular distribution dynamics of C′-dots in brain tumors as a function of blood-brain permeability, time, RGD targeting and drug conjugation using a genetically-engineered mouse glioma model, and (2) determining the pharmacologic efficacy and optimized dosing of C′-dots conjugated to small molecule EGFR inhibitors via cleavable linkers in a metastatic model of EGFR-mutant non-small cell lung cancer.

Following incubation of EGFRmt+ and PDGFB-driven tumor cell lines with gefitinib (or dasatinib)-modified C′ dots, cellular internalization, inhibitory profiles, and viability were evaluated over a range of particle concentrations and times (i.e., 6, 18 hrs) relative to native SMIs. Regarding EGFRmt+ expressing cell lines, non-small cell lung cancer (NSCLC) lines were tested, including L858R ECLC26, a line containing an activating single-point substitution mutation L858R in exon 21, which confers sensitivity to EGFR tyrosine kinase inhibitors. A less sensitive NSCLC line was also used, H1650, which harbors resistance mutations. For PDGFR-expressing cells, 3T3 cells and PDGFB-driven primary cells were used. In the latter case, cells were derived from a genetically engineered mouse model (GEMM) of high-grade glioma using RCAS for PDGF-B gene transfer while genetically engineering its receptor, tv-a, into strains of mice under the GFAP or nestin promoters (i.e., Gtv-a and Ntv-a, respectively). EGFR and PDGFR phosphorylation status of cells were assayed by western blot, and findings used to select lead candidates for in vivo efficacy studies.

In parallel with in vitro studies, in vivo baseline studies were performed using the base particle probe (i.e., FDA-IND approved cRGDY-PEG-C′ dots) and dasatinib-NDCs in conjunction with time-dependent intravital staining methods to provide initial assessments of intratumoral penetration and particle distribution kinetics as a function of blood-brain barrier permeability, integrin-targeting (vs non-integrin targeting using cRADY-PEG-C′ dots) and, subsequently, drug conjugation in RCAS-tva GEMM of high-grade glioma.

Dose escalation studies with the dual-modality particle probes are being used to investigate improvements in targeted therapeutic delivery, penetration, and maximum treatment response over the native drug for both dasatinib-NDCs in PDGFB-driven gliomas and gefitinib-NDCs in EGFRmt+ preclinical flank/brain xenograft models; imaging findings are being confirmed histologically. Pharmacokinetic studies have also been performed with these agents to assess for unexpected toxicity and evaluate particle dosimetry. A separate cohort of mice can be injected with dual-modality particle probes to track drug vs particle delivery and distribution to monitor stability of the platform. Expected increased effective drug concentrations at tumor sites are based upon previously observed preferential tumor retention and the ability to quantitatively estimate therapeutic dosing requirements for tracer NDC doses using PET imaging.

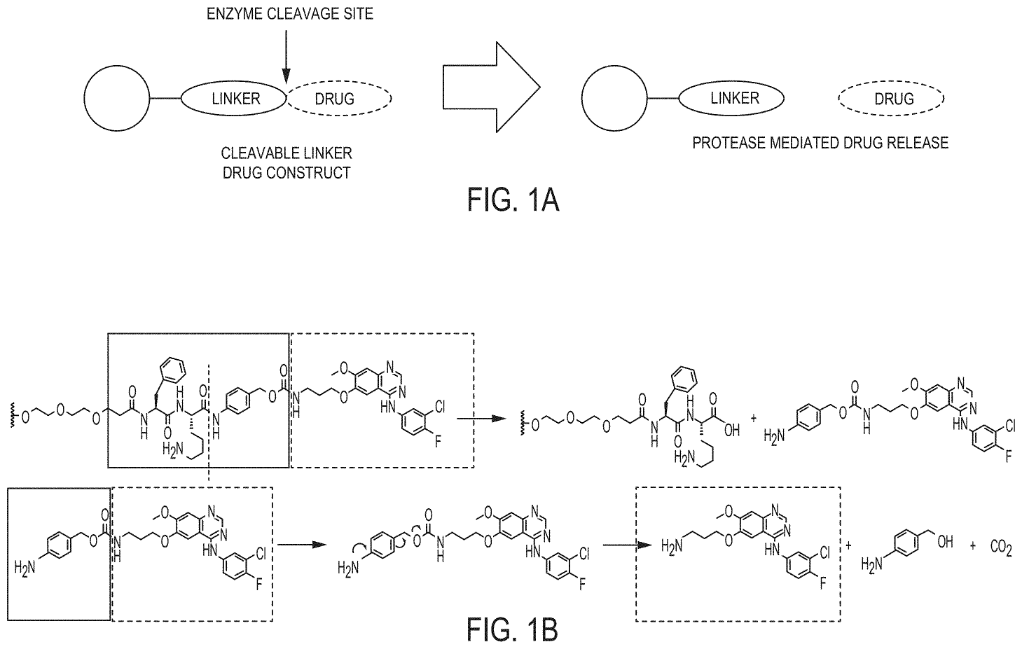

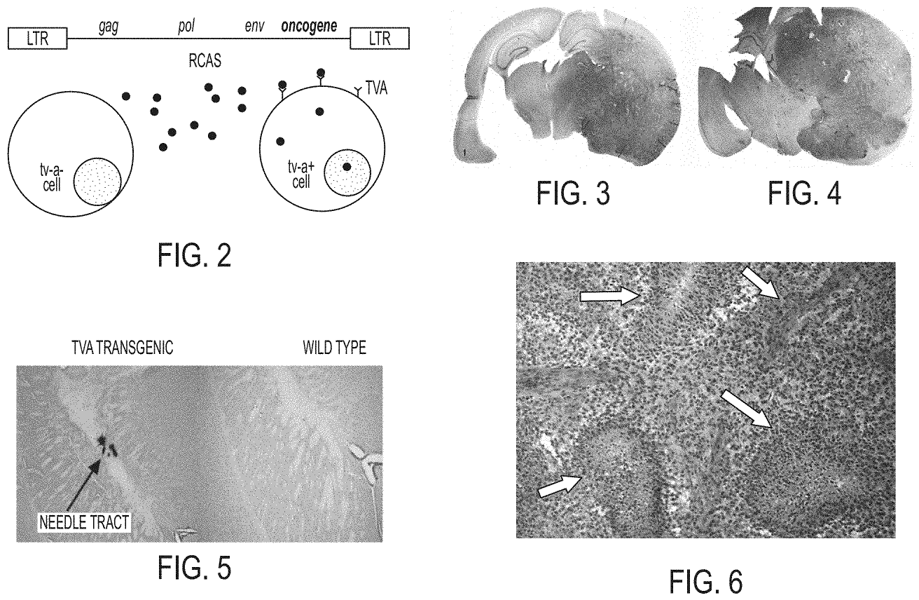

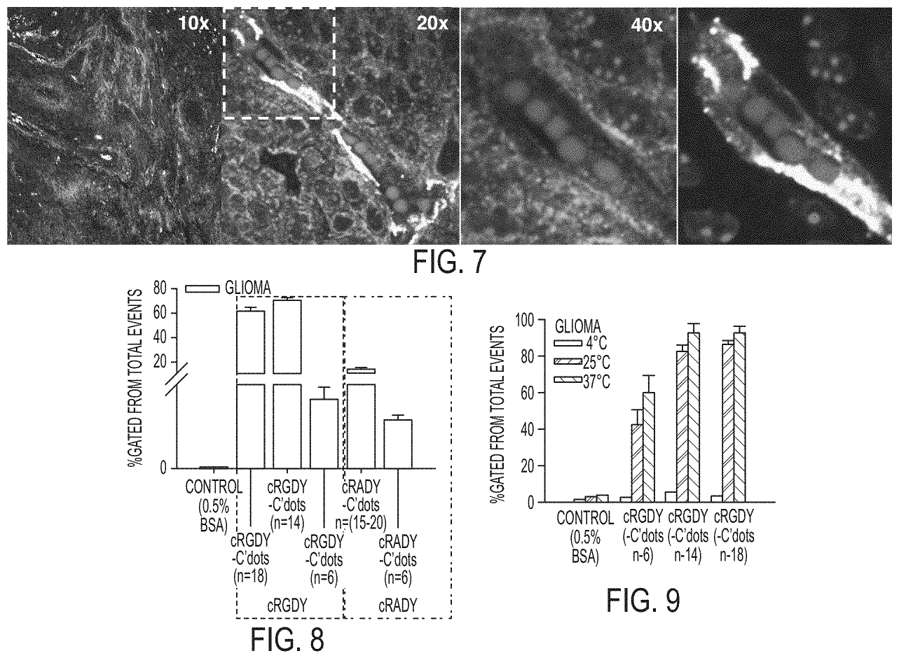

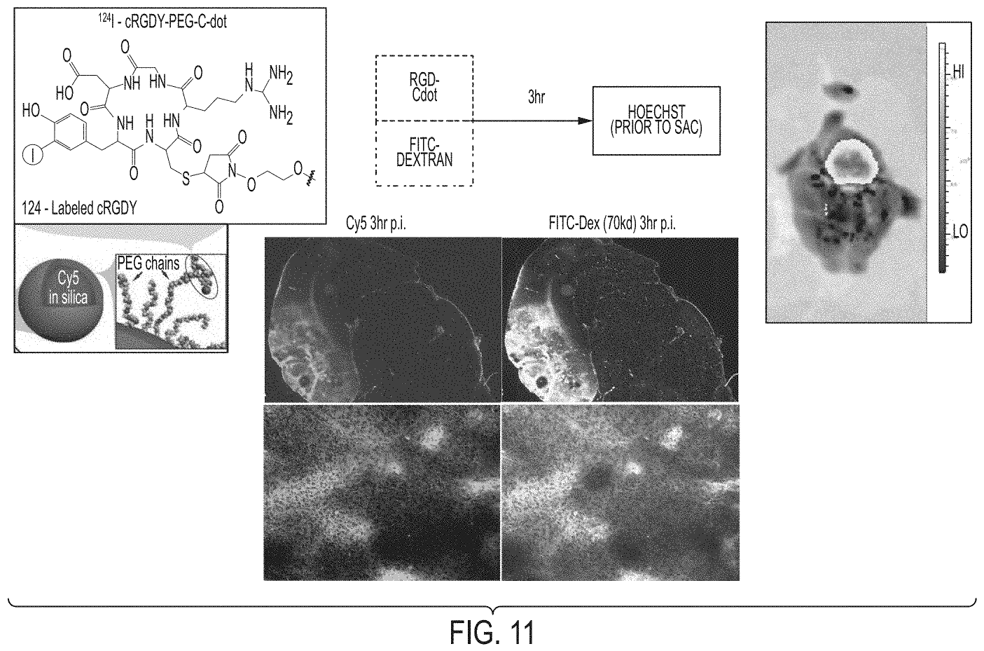

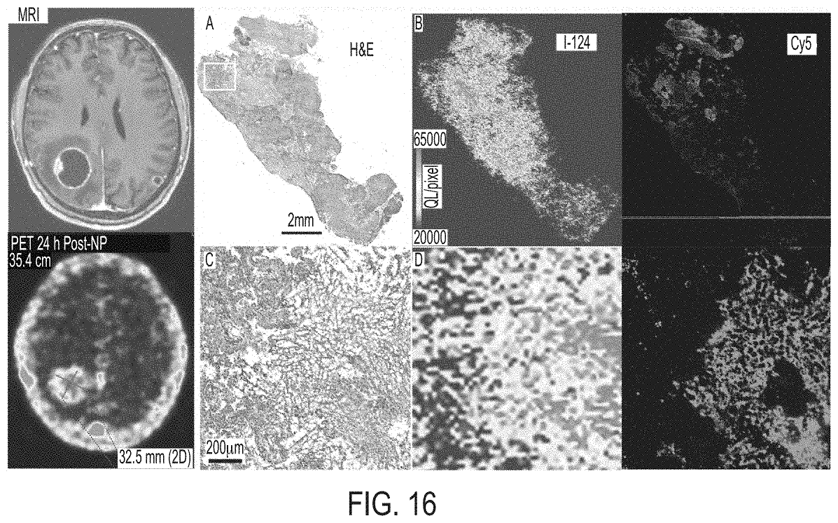

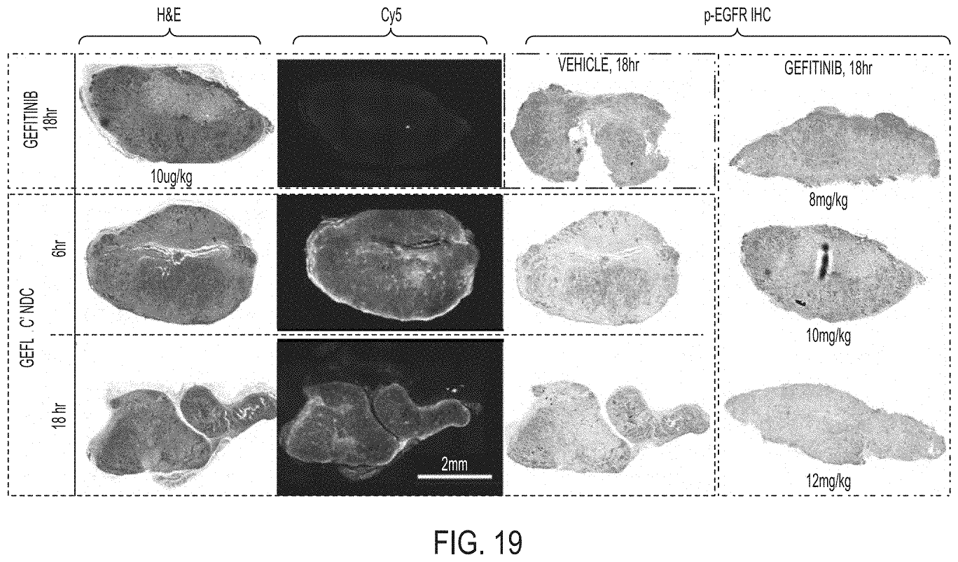

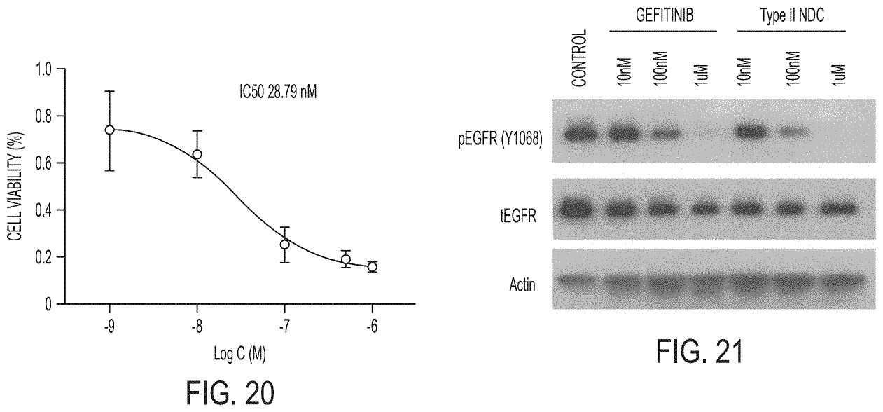

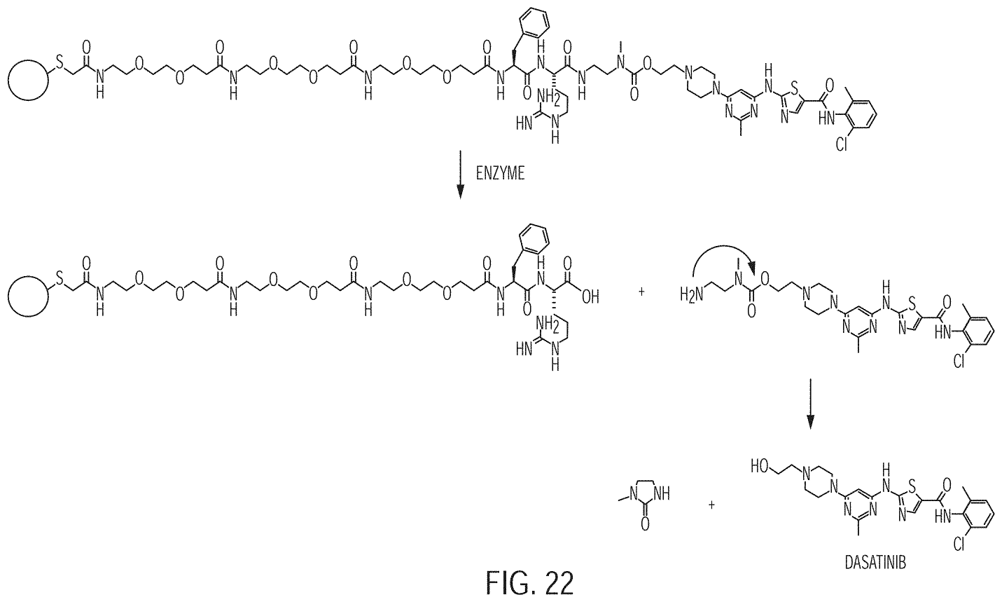

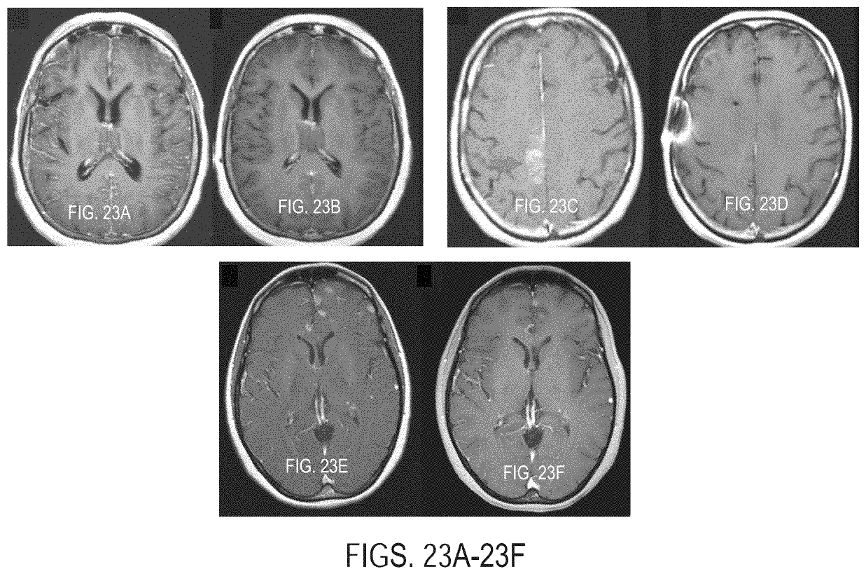

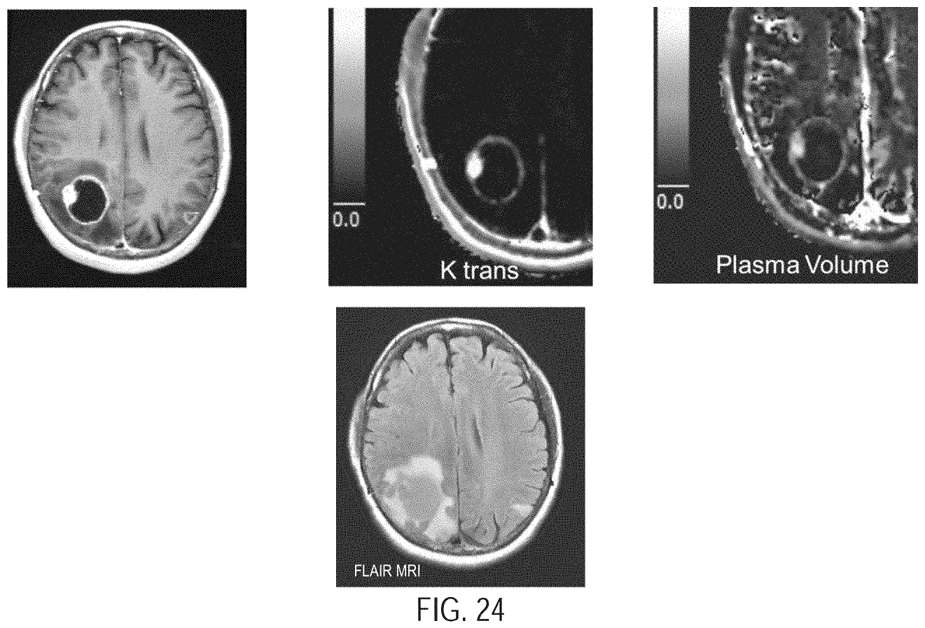

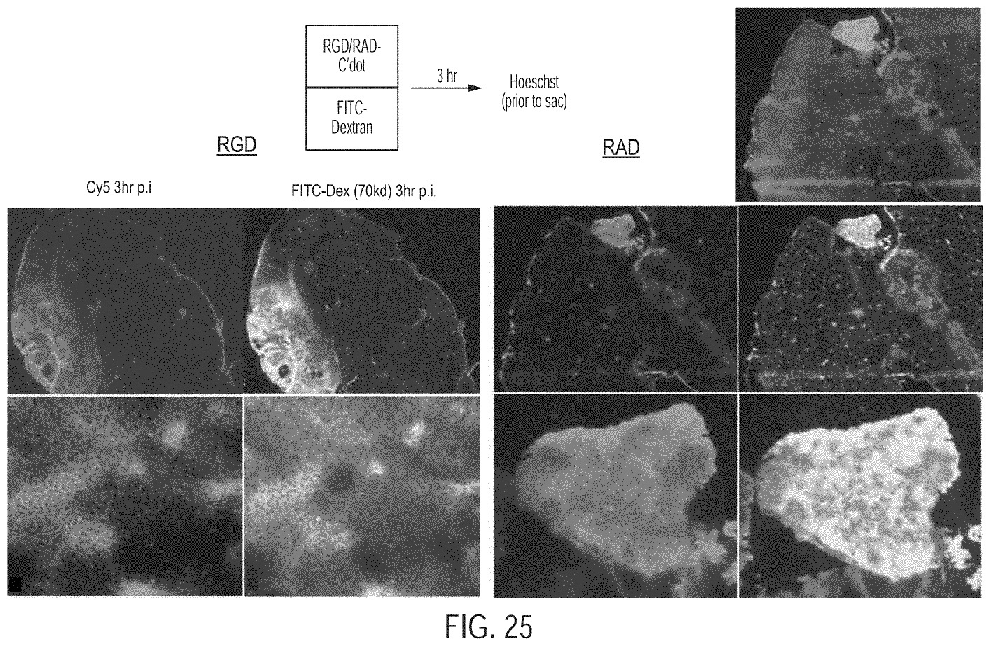

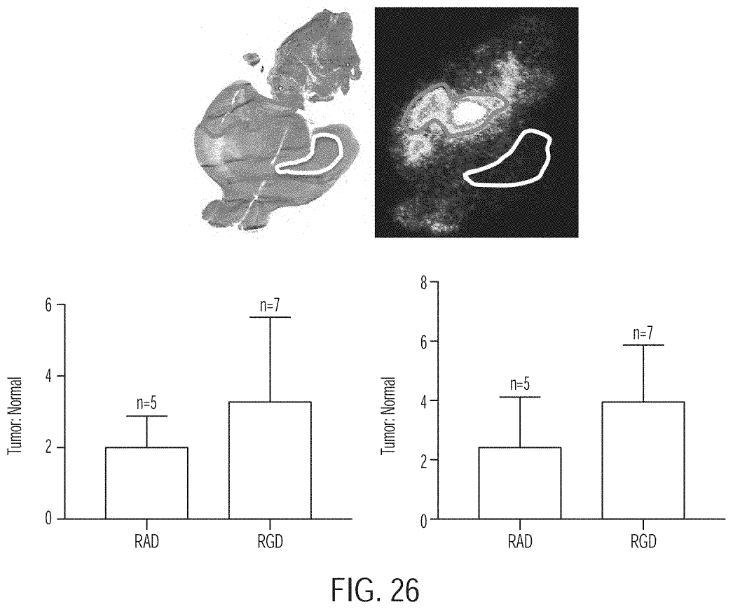

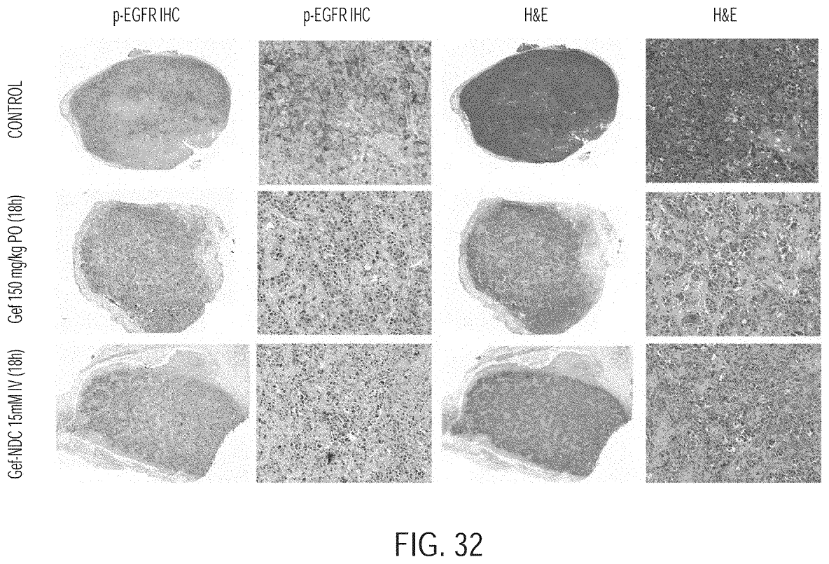

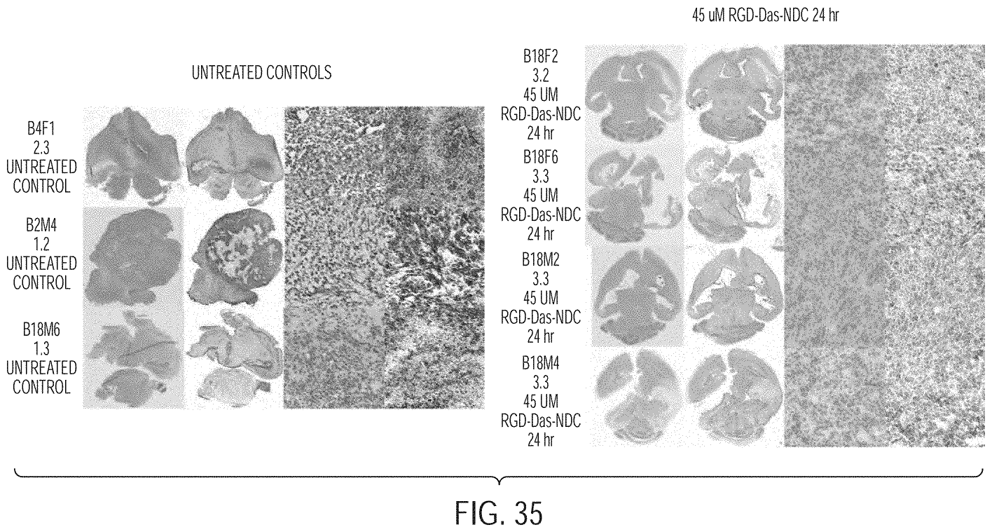

These SMI-bearing platforms have also been further adapted with targeting peptides, including cRGDY and αMSH, the former for delivering and targeting SMIs to integrin and/or melanocortin-1 (MC1) receptors. Integrins are expressed by primary glioma cells and by tumor vascular endothelial cells, while the latter is expressed by tumor-associated macrophages in the microenvironment. The contribution of integrin receptor targeting to the overall intratumoral accumulation of these probes can then be determined for this ultrasmall (sub-10 nm) particle size. Non-specific uptake in tumors due to enhanced permeability retention (EPR) effects can also be assessed using scrambled peptide (cRADY)-bound C′ dots (controls), which do not bind to integrin receptors. Without wishing to be bound to theory, it is believed that the ultrasmall size of these particles enables diffusion within the tumor interstitium (see FIGS. 1-35) to reach a larger number of cellular targets, as against larger nanomaterials (i.e., liposomes), which largely accumulate along vessel walls at the site of vascular leakage (via the EPR effect). Such theranostic platforms (diagnostic-therapeutic) can be used to treat targets in both the tumor and surrounding microenvironment (via macrophages or other immune/inflammatory cell types). For example, while dasatinib may be used on cRGDY-bound C′ dots to target primary glioma cells (and activated endothelium), inhibitors for targeting TAMs (i.e., inhibitors of the macrophage CSF-1 receptor (CSF-1R)) or other immune components may be attached to αMSH-bound C′ dots. It should be noted that αMSH is a neuroimmunomodulator, and its receptor, MC1-R, is present on macrophages.

Preclinical study results are being used to inform clinical trial designs. Targeted delivery and penetration of ¹²⁴I-cRGDY-bound-C′ dots are currently being monitored in pre-surgical patients harboring either brain metastases (i.e., NSCLC, breast cancer) or GBM, two tumor types for which improved delivery of SMIs to the CNS is likely to be clinically significant. Following intravenous injection of ¹²⁴I-cRGDY-bound C′ dots, serial PET-CT imaging is being used to detect, localize, and assess accumulations of the particle tracer within brain tumors over a 24 hour period. To correlate imaging with molecular abnormalities and tissue particle distributions, tissue is being analyzed from tumor biopsies targeting regions of tracer uptake within and about the tumor. The experimental protocol involves: (1) preoperative MRI per routine and PET-CT imaging p.i. ¹²⁴I-cRGDY-PEG-C′ dots co-registered for identification of potential biopsy target/s; (2) surgical resection with targeted tissue acquisition per routine, with integrated frameless stereotactic tracking used to annotate sites of biopsies, and updated by intraoperative MRI (iMRI, 1.5 T Siemens magnet). Tissue samples from several regions are collected within and around the tumor. Tumor tissue regions showing particle tracer uptake and other tissue showing little or no uptake are being analyzed for integrin expression. Assays include immunohistochemistry with commercially available antibodies.

Furthermore, it is contemplated that the conjugates described herein can be used to manipulate (e.g., regulate, control) behavior of certain cells (e.g., macrophages, tumor-associated macrophages and/or microglia (TAMs), dendritic cells, and/or T cells) in a tumor microenvironment (e.g., in vivo, e.g., in the treatment of cancer, e.g., brain cancer, e.g., malignant cancer, e.g., malignant brain cancer), for improved treatment efficacy. For example, a conjugate of an ultrasmall nanoparticle with an inhibitor of CSF-1 receptor (CSF-1R) can be used to target tumor-associated macrophages in a tumor microenvironment for their regulation/control in the treatment of the tumor. For example, the described nanoparticle conjugates can comprise a modulator moiety (e.g., an inhibitor of colony stimulating factor-1 (CSF-1R) for targeting TAMs.

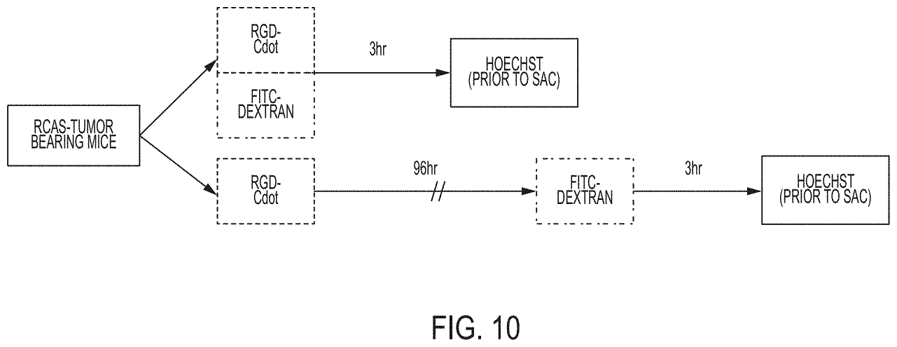

A chart illustrating use of the RCAS-PDGFB mouse glioma model to study C′-dot distribution via concurrent intravital staining is shown in FIG. 10. To further evaluate how this particle may be used therapeutically, particle distribution was further investigated, both within the tumor and on a cellular level. Using the RCAS brain tumor model, a methodology to administer fluorescent labels prior to sacrifice was developed. Hoechst was used to stain cell nuclei as a marker of cellular localization, and a green fluorescent FITC-70 kDa dextran was used to roughly approximate the size of the particle as marker of blood brain barrier breakdown and to estimate the EPR effect alone on a small dextran. The particle distribution over time was also investigated, looking at a short 3 hour post-treatment timepoint compared to a 96 hour time point.

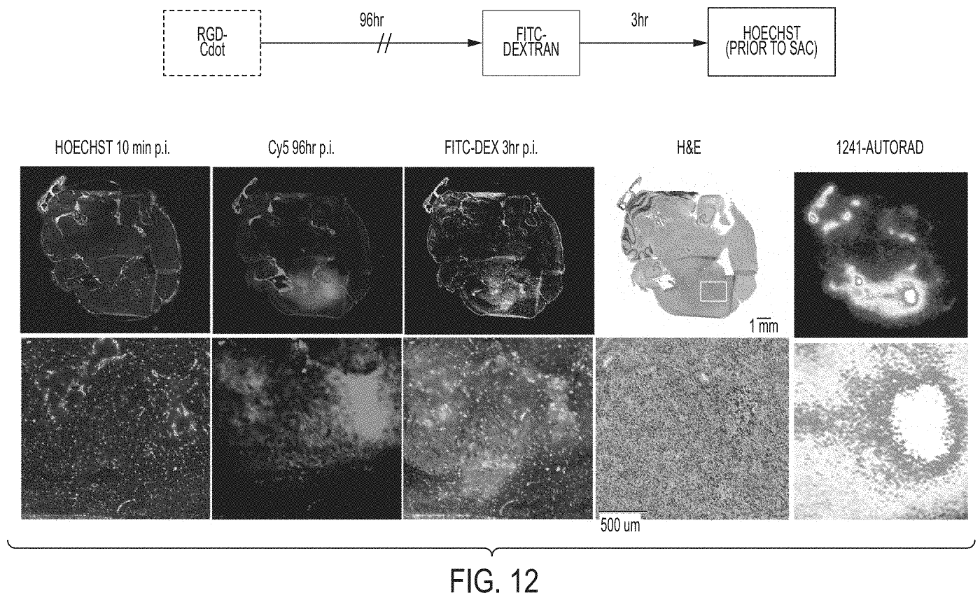

Images from an ex vivo study of ¹²⁴I-RGD-Cy5-C-dot distribution in RCAS tumor-bearing mice are also provided in FIG. 12. RGD-targeted nanoparticle is strongly retained in tumor at 96 h post-injection (p.i) and diffuses beyond 70 kDa Dextran given 3 h prior to sacrifice. RCAS-tva tumor bearing mice are treated in vivo with RGD-targeted Cdots 96 h prior to sacrifice (p.t.s.), FITC-Dextran 3 h p.t.s, followed by Hoechst 10 minutes p.t.s. Compared to the close approximation of Cy5 and FITC signal when co-administered 3 h p.t.s., Cy5 signal 96 h post-treatment appears more diffuse than the FITC signal in concentrated regions of BBB breakdown within the tumor, and retains high signal intensity. The Cy5 signal closely approximates the regions of tumor as identified on H&E. The RGD-targeted Cdot is retained at 96 hours and appears to diffuse through the tumor beyond regions of BBB breakdown alone. I-124 autoradiography demonstrates illumination in region closely matching Cy5 signal, suggesting that I-124 remains attached to the Cy5 containing Cdot in vivo.

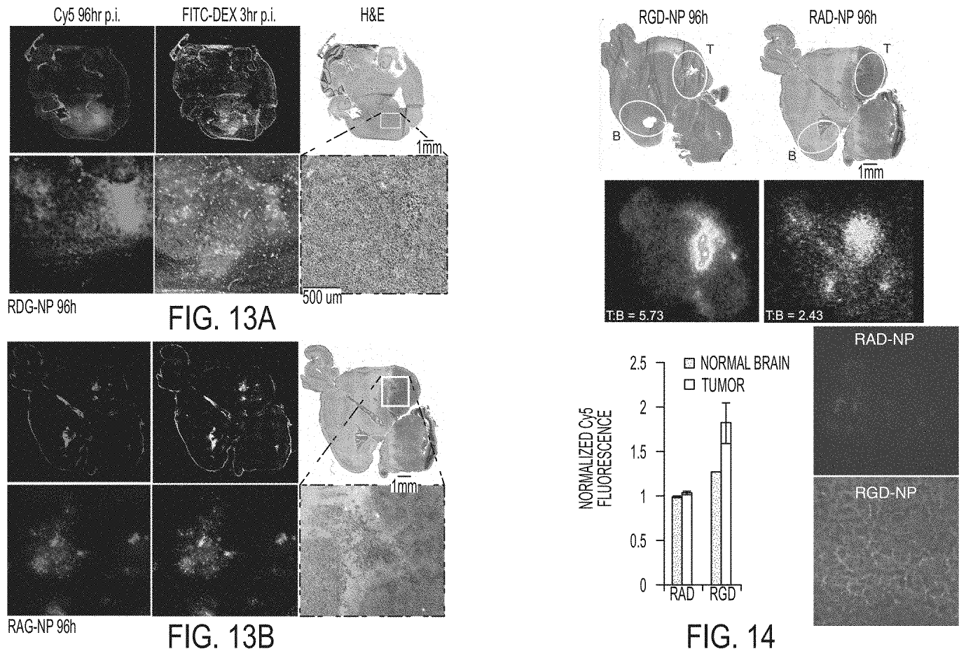

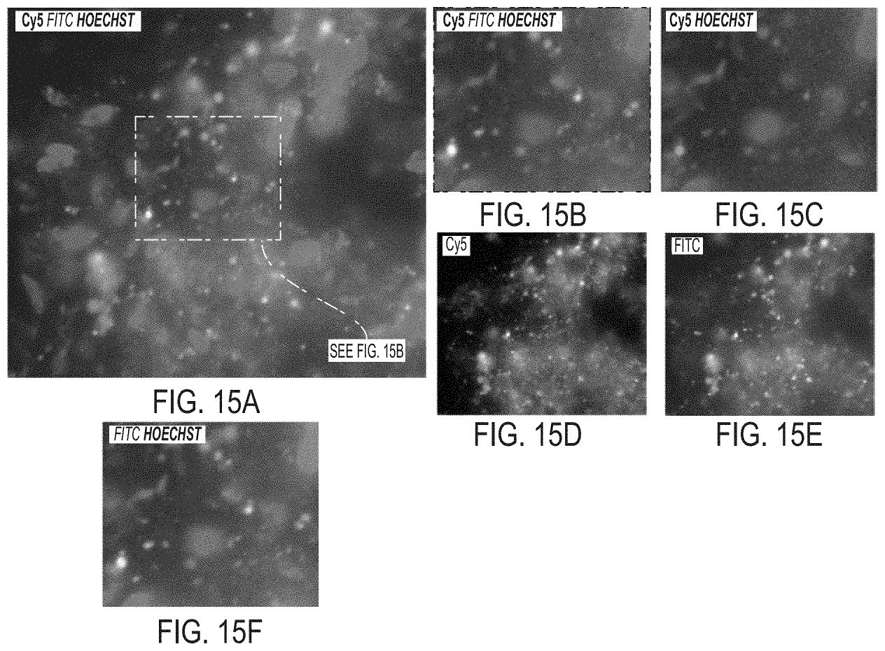

Triple fluorescence labeling images of FITC-Dextran as a reference tracer of similar size to the nanoparticle conjugates of FIGS. 13A, 13B, and 14 are shown in FIGS. 15A-15F. As explained above, the data is suggestive of intracellular localization of Cy5-C′ dots at least as early as three hours post-treatment. In addition to tumor distribution, high-magnification imaging of the tumor sections were taken to visualize particle distribution on the cellular level. In these images, a strong nuclear stain in blue surrounded closely by nanoparticle in red is seen. Without wishing to be bound to any theory, this data is suggestive of intracellular localization, possibly in lysosomes.

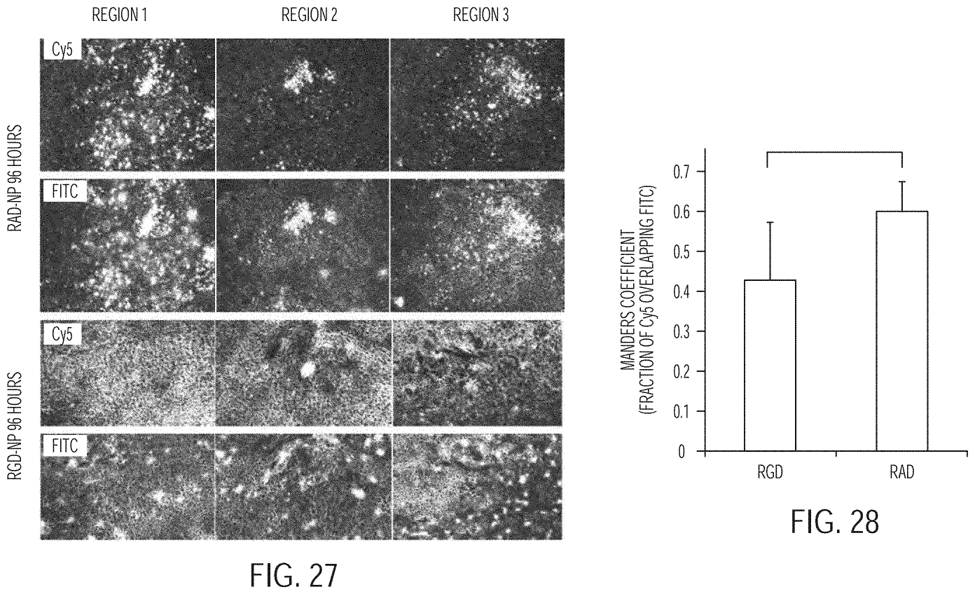

FIGS. 27 and 28 shows images and data depicting distribution analysis by pixel correlation. High-powered imaging of focal regions of tumor treated with targeted and non-targeted nanoparticle. RCAS-tva tumor bearing mice were treated with either radiolabeled RGD-targeted nanoparticle or RAD-nanoparticle and sacrificed at 96 hours post-treatment with injection of FITC-Dextran 3 hours prior to sacrifice. Frozen sections were analyzed for fluorescent signal using high-powered imaging of representative regions. The data demonstrates closely overlapping regions of RAD-nanoparticle signal with regions of BBB breakdown as marked by FITC, compared to a more diffuse pattern of Cy5 signal beyond FITC hotspots in RGD-nanoparticle treated tumors.

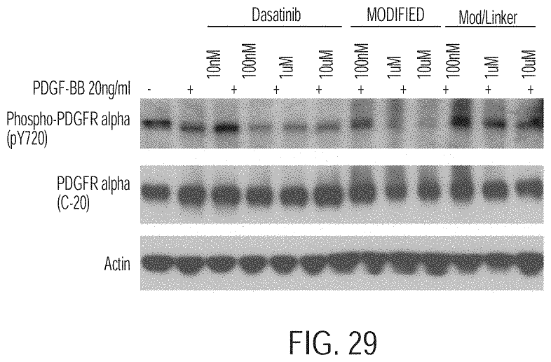

FIG. 29 shows an image of a western blot indicating that dasatinib-NDC achieves PDGFR inhibition in a dose-dependent manner at levels similar to free drug. TS543 (Neurosphere cells) were treated with indicated drugs for 4 hours followed by PDGF-BB 20 ng/ml for 10 minutes. Cells were starved in stem cell medium without growth factors for 18 hours before treatment. The modified/linker Dasatinib-NDCs demonstrated p-PDGFR α inhibition in a dose-dependent fashion at doses comparable to doses demonstrating p-PDGFR α inhibition by free Dasatinib.

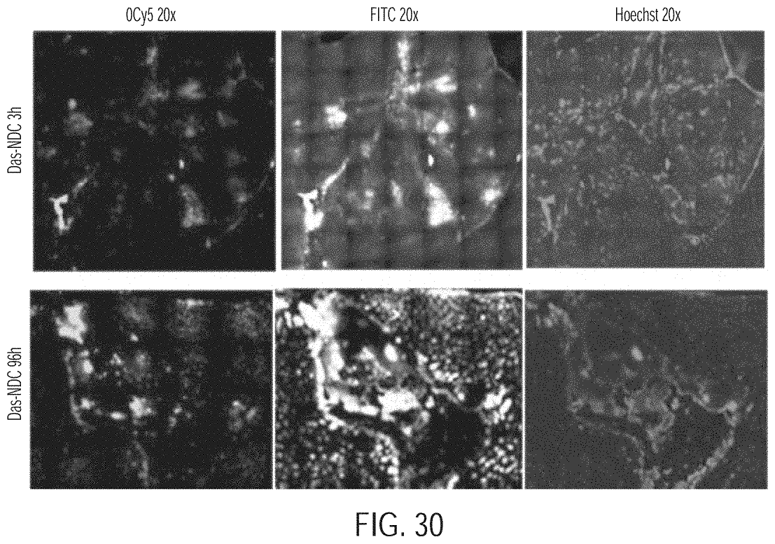

FIG. 30 shows images of dasatinib-NDC distribution in tumor at 3 and 96 hours post-treatment. RCAS-tva tumor bearing mice were treated with non-targeted Dasatinib-nanoparticle conjugate (Das-NDC) and sacrificed at 3 and 96 hours post-treatment with injection of FITC-Dextran 3 hours prior to sacrifice. Frozen sections were analyzed for fluorescent signal using high-powered imaging of representative regions. High degrees of overlap were seen between Cy5 and FITC signal at 3 and 96 hours, replicating similar findings in the corresponding non-targeted unconjugated nanoparticle (RAD-NP).

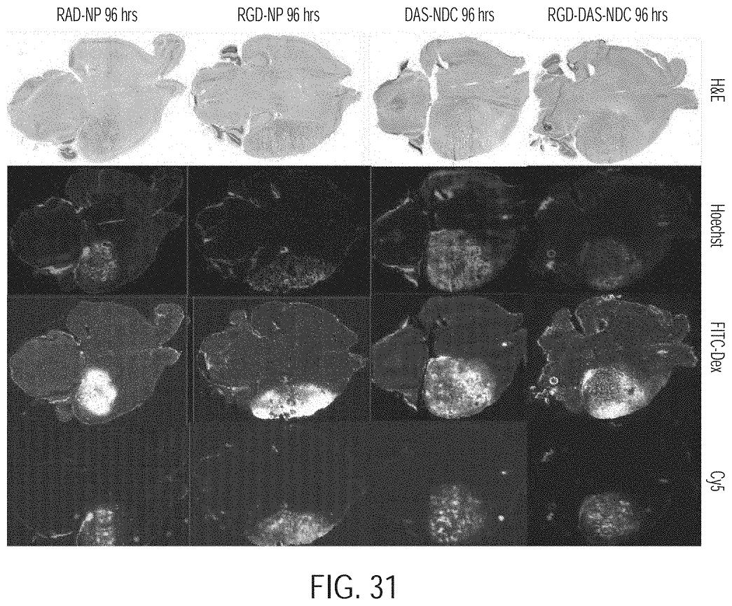

FIG. 31 shows H&E and fluorescence images of comparable distribution of fluorescent signal in targeted and non-targeted nanoparticle-drug conjugate compared to targeted and non-targeted particle alone. RCAS-tva tumor bearing mice were treated with non-targeted Dasatinib-nanoparticle conjugate (Das-NDC) and targeted Dasatinib-nanoparticle conjugate (RGD-DAS-NDC) and sacrificed at 96 hours post-treatment with injection of FITC-Dextran 3 hours prior to sacrifice and Hoechst 10 minutes prior to sacrifice. Frozen sections were analyzed for fluorescent signal using high-powered imaging of representative regions. Representative samples demonstrating similar distribution in the non-targeted Das-NDC tumors compared to RAD-NP, as well as RGD-NDC compared to RGD-NP, suggesting retention of nanoparticle uptake and diffusion properties with the introduction of the drug conjugate.

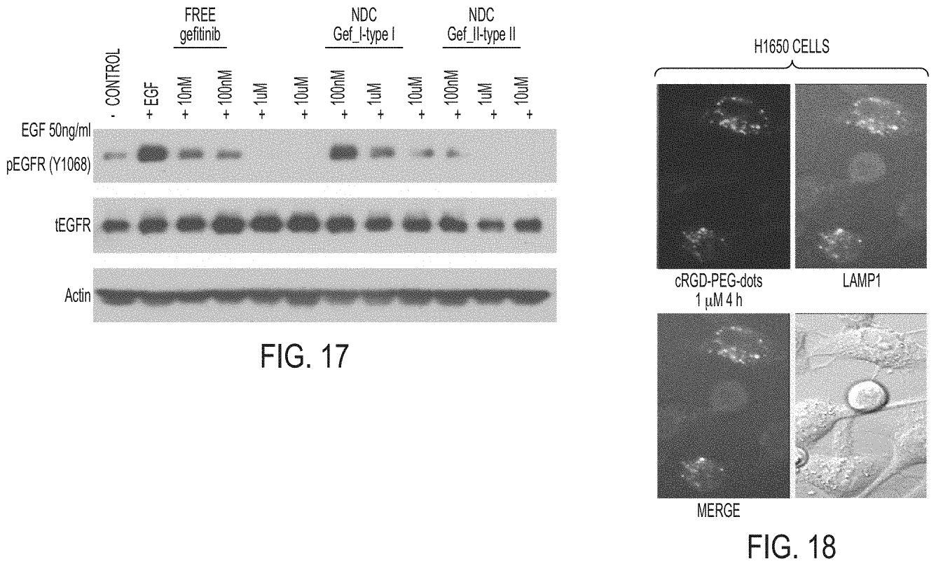

In order to study drug-conjugate kinetics, SMIs were used as a model system. A gefitinib drug model, which has efficacy in the primary NSCLC but not in the treatment of brain metastases, was used, and its properties of being highly protein bound and hepatically cleared are shown in FIGS. 17 and 18. If nanoparticle kinetics can improve on this with enhanced renal clearance, a higher therapeutic index can be achieved. Accordingly, gefitinib was attached to the C′-dot using optimization of drug-linker combinations. It was then demonstrated that the modified drug-NP conjugate retained potency as measured by pEGFR inhibition despite drug modifications. Optimization of delivery and release of the SMI from NDCs can be investigated.

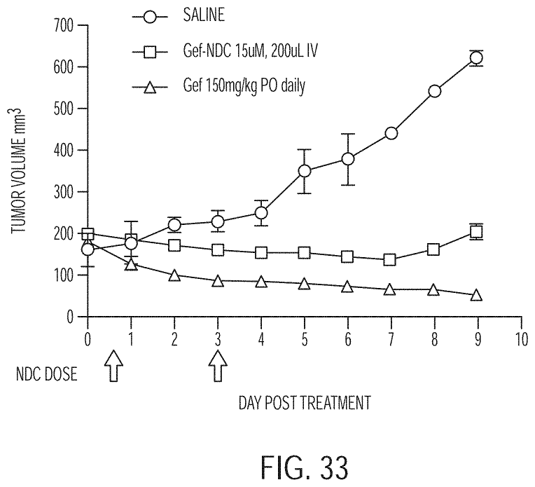

FIG. 33 shows data from a multi-dose treatment of ECLC26 flank tumor bearing mice that results in robust tumor control. Analyzation of growth was seen at days 8 and 9. ECLC 26 tumor-bearing mice were treated with either Gefitinib-NDC at two time points, daily Gefitinb P.O. (150 mg/kg), or daily oral saline vehicle in a multi-dose model with dose administration indicated by the blue arrows. Tumor volume was measured using caliper measurements of the maximal dimensions of the tumor daily. This graph demonstrates the natural growth of the vehicle-treated tumor over time compared to the steady decrease in tumor volume in the Gef-NDC and Gefitinib treated groups. Notably, there is recovery of tumor growth in the Gef-NDC treated group around 8 days post-treatment, suggesting possible attenuation in efficacy at this time point.

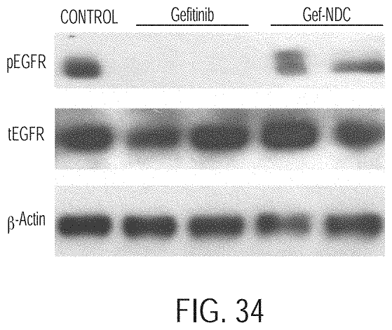

FIG. 34 shows ECLC26 growth post treatments. Nude mice were implanted with 2 million ECLC26 cells. Mice bearing tumors were treated by i.v. of 200 μL saline or 15 μM Gef-NDC for 2 doses of Gavage with 15 mg/ml Gefitinib, 10 μL/g for 10 days.

### Example 2: Regulating the Tumor Microenvironment with Targeted Ultrasmall Silica Nanoparticle Imaging Probes (C′ dots) for Small Molecule Inhibitor Delivery and Imaging

Therapeutic approaches targeting high-grade glioma have largely failed. An alternative strategy is to regulate cells, such as tumor-associated macrophages and microglia (TAMs), in the tumor microenvironment (TME). TAMs account for as much as 30% of the tumor mass in mouse models of high-grade glioma and in brain tumor patients; TAM accumulation is associated with higher glioma grade and poor patient prognosis. Colony stimulating factor-1 (CSF-1) is known to influence differentiation and survival of macrophages, as well as their activation or polarization state. In a PDGF-driven mouse glioma model, inhibition of CSF-1R has been shown to suppress the M2 phenotype, to reduce tumor growth, and improve survival.

The present Example selectively delivers small molecule inhibitors, such as the CSF-1R agent BLZ945, to TAMs by attaching synthesized drugs and targeting peptides, for instance, alpha melanocyte stimulating hormone (αMSH), to ultrasmall fluorescent silica nanoparticles (C′ dots). Such compositions are referred herein as “nanoparticle drug conjugates (NDCs)”. By using a PDGF-driven mouse glioma model with established sensitivity to TAM regulation, targeted delivery and efficacy of this NDC was assessed and compared with the established efficacy of BLZ945 as a free drug. Moreover, combination treatments with integrin-targeted NDCs incorporating the PDGF inhibitor, dasatinib, were evaluated.

TAMs are the most prevalent inflammatory cell in the TME where they comprise a heterogeneous community of distinct functional subtypes. Although the range of TAM phenotypes is not completely understood, activated TAMs expressing markers of an M2 class have been shown to contribute to tumor initiation and maintenance, as well as influence anti-tumor autoimmunity via cytokine release and inflammatory recruitment in the TME. Tumors, in turn, can promote the polarization of monocytes into M2 TAMs by releasing factors, such as TGF-beta and M-CSF. The therapeutic regulation of TAM subtypes through intact physiologic mechanisms is a potentially potent means to influence the TME in a broad range of cancers.

As described herein, targeting of TAMs in cancer can be most effective when combined with other therapies directed at tumor cells. Indeed, the first trial of CSF-1R inhibition as a monotherapy in glioma found little efficacy.

As described herein, ultrasmall nanoparticles (e.g., C′ dots) were used to selectively deliver a receptor tyrosine kinase (RTK) inhibitor (e.g., BLZ945), to melanocortin-1 receptor (MC1R) expressing TAMs by attaching its ligand, alpha melanocyte stimulating hormone (αMSH), a neuroimmunomodulator. BLZ945, a specific CSF-1R inhibitor that regulates macrophage polarization and function, was synthesized and modified for attachment to C′ dots as described in International Application No. PCT/US2015/032565 (published as WO 2015/183882 on Dec. 3, 2015), the contents of which are hereby incorporated by reference in its entirety.

The exquisite brightness of the resulting NDCs was exploited to assess uptake of αMSH-targeted particles in macrophages in vitro and in tumors, utilizing RCAS PDGFB-driven genetically engineered mouse models (GEMM) of high-grade glioma. This model was chosen due to its sensitivity to TME regulation by CSF-1R inhibition, as well as its disruption of tumor cell signaling by the PDGF and Src inhibitor, dasatinib (das). As such, the efficacy of particle-based delivery of these drugs, singly and potentially in combination, was tested. Development of das-RGDY-PEG-C′ dots provides for methodologies for mapping delivery and diffusion of das-RGDY-PEG-C′ dots and BLZ947-αMSH-PEG-C′ dots as a function of blood-brain-barrier permeability.

### Synthesis and Characterization of Targeted NDCs as Combinatorial Agents to Independently Target Tumor Cells and TAMs in High-Grade Gliomas

Two RTK inhibitors, BLZ945 and dasatinib (das), were conjugated onto C′ dots through the use of cleavable chemical linkers. BLZ945, a CSF-1R specific RTK inhibitor developed at MSKCC, was adapted with a dipeptide based chemical linker. This drug-linker construct was conjugated onto αMSHPEG-C′ dots to form NDC BLZ945-αMSH-PEG-C′ dots for targeting TAMs, while dasatinib was conjugated onto cRGDY-PEG-C′ dots for targeting integrin-expressing glioma cells. An alternate strategy is to conjugate the CSF-1R multikinase inhibitor, PLX3397, if modification of BLZ945 impairs CSF-1R inhibition.

Synthesis of das-cRGDY-PEG-C′ dots are also provided. For example, a modified dasatinib analog that has been conjugated via cleavable linker to C′ dots is also provided. A das analog was conjugated onto cRGDY functionalized particles to form the NDC das-cRGDY-PEG-C′ dot. Moreover, characterization of BLZ945-αMSH-PEG-C′ dots and das-cRGDY-PEG-C′ dots was performed via HPLC methods to assess drug load.

Drug release in the presence of serine and cysteine proteases (e.g., trypsin and cathepsin) was evaluated by liquid chromatography-mass spectrometry.

Assessment of C′ Dots Adapted with One or More Targeting Moieties (BLZ945; αMSH) to Activate CSF-1R and MC1R Expressing TAMs by Evaluating Cytokine Secretion and Gene Signatures.

RAW 264.7 mouse macrophages and primary mouse bone marrow-derived macrophages (BMDM) were cultured in U-251 glioma conditioned media (GCM), which protects macrophages from BLZ945-induced cell death. BMDM was prepared and cultured. Moreover, a chelator-free radiolabeling strategy was compared with traditional chelator-based radiolabeling methods for particle radiolabeling in terms of stability, radiochemical yield, specific activity, tumor target uptake, and tumor-to-background ratios. The chelator-free approach relies on ⁸⁹Zr labeling of intrinsic C′ dot deprotonated silanol groups (—Si—O—); chelator-based methods include conjugation of glutathione and desferrioxamine B to C′ dot surface-bound PEG chains prior to ⁸⁹Zr labeling.

Competitive binding studies were performed for ⁸⁹Zr-αMSH-bound NDCs, as against the native ‘cold’ TKI, using MC1-R expressing macrophages and gamma counting detection methods to determine binding affinity and potency. To examine binding specificity, MC1-R blocking experiments were conducted using anti-MC1R antibody prior to particle exposure and flow cytometry. Intracellular trafficking of particles through the endocytic pathway and lysosomal uptake were also examined. To investigate trafficking of C′ dots through the endocytic pathway, fluorescent reporters of endocytic trafficking were expressed, and colocalization with ingested targeted NDCs and particle controls were examined by time-lapse microscopy.

Macrophages were incubated with BLZ945-conjugated αMSH-PEG-C′ dots to inhibit CSF-1R signaling, and to target macrophages through the αMSH ligand which binds MC1R expressed on these cells. Dose- and time-dependent particle uptake into macrophages, cultured in control or U-251 glioma conditioned medium, was quantified by flow cytometry and fluorescence microscopy. Cell viability was assayed by standard MTT assays following particle exposure (BLZ945-αMSH-PEG-C′ dots, BLZ945-PEG-C′ dots, αMSH-PEG-C′ dots, PEG-C′ dots). If particle treatments are well tolerated under these conditions, their effects on macrophage function can be examined.

As treatment with BLZ945 has been shown to influence the activation or polarization state of TAMs (e.g., decreased expression of M2 polarization markers), the effects of BLZ945-conjugated-αMSH-PEG-C′ dots on macrophage polarization and function were examined. Positive results stemming from these initial studies, which suggest that BLZ945-conjugated-αMSH-PEG-C′ dots affect macrophage function in a manner similar to soluble BLZ945, were used to guide further testing with αMSH-PEG-C′ dots lacking the CSF-1R inhibitor or PEG-C′ dots lacking the αMSH targeting ligand, to determine whether the base particle may also contribute to modulating macrophage properties.

RAW 264.7 macrophages and BMDM, cultured in GCM, were exposed to escalating doses of BLZ945-αMSH-PEG-C′ dots or soluble BLZ945 (at 670 nM), and examined for expression of a four-gene signature (Adrenomedulin, Arginase 1, Clotting factor F13-a1, Mannose receptor). Cytokines associated with M1 (e.g., TNFα, IL-12P70, IL-10, IFN-γ) or M2 polarization (e.g., IL-10, TGFβ) were evaluated by ELISA-based detection from culture medium. Target gene expression of early growth receptor 2 (Egr2), a transcription factor downstream of CSF-1R, can be quantified in control and treated cells by QRT-PCR to determine the extent of inhibition of CSF-1R activation by particle treatments. Modulation of the phagocytic activity of cultured macrophages, a hallmark of M1 polarization shown to be upregulated by BLZ945 treatment, was examined by incubating RAW 264.7 or BMDM with apoptotic cells and quantifying phagocytic index.

### Evaluation of Binding/Uptake Properties and Specificity of Das-cRGDY-PEG-C′ Dots

Competitive binding studies were performed to assess binding affinity and potency of ⁸⁹Zr-das-cRGDY-PEG-C′ dots as against the native ‘cold’ TKI (das) using the described methods, except using primary cells derived from PDGFB-driven gliomas. Integrin receptor blocking studies were conducted using an anti-αv integrin antibody prior to particle exposure. Viability studies were conducted following particle exposure (das-cRGDYPEG-C′ dots, das-PEG-C′ dots, cRGDY-PEG-C′ dots, PEG-C′ dots) using methods described herein.

### Quantitative Assessment of PK Profiles and Tumor-Selective Accumulations

Quantitative assessment of PK profiles and tumor-selective accumulations of ⁸⁹Zr-labeled peptide-bound NDCs (e.g., BLZ945-αMSH-PEG-C′ dots, das-cRGDY-PEG-C′ dots) relative to ⁸⁹Zr-NDCs (e.g., ⁸⁹Zr-BLZ945-, ⁸⁹Zr-das-PEG-C′ dots) and ⁸⁹Zr-labeled particle controls (αMSH-, cRGDY-PEG-C′ dots) in PDGFB-driven highgrade glioma models with histologic correlation are described.

Gliomas were generated by RCAS-mediated transfer of the oncogenic driver PDGFB to nestin+ progenitor cells in the brain of Nestin-tva mice. Following intravenous (i.v.) injection of ⁸⁹Zr-αMSH (or ⁸⁹Zr-cRGDY) NDCs, ⁸⁹Zr-NDCs, or ⁸⁹Zr-labeled particle controls (˜20 μCi/mouse), glioma mice (n=5 per particle) were sacrificed at 5 specified time points (4 h-168 h), and blood, urine, tumor, and organs can be harvested, weighed, and gamma counted to determine % ID/g, corrected for decay to time of injection. Results were compared with those of respective particle controls. RadioTLC of blood and urine were also conducted to assess particle stability over this interval.

As described herein, serial 15 min static images were acquired on the Inveon PET/CT scanner over 96-hour intervals after i.v. injection of 200 μCi of ⁸⁹Zr-labeled peptide-bound NDCs, non peptide-bound NDCs, and control probes using separate cohorts of mice.

Histologic assays, digital autoradiography, and multichannel fluorescence microscopy of resected tumor tissue specimens were performed to evaluate and compare intracellular localization and particle distributions among imaging particle probes.

### Determination of Whether Improved Therapeutic Efficacy is Achieved for Targeted NDCs Relative to Particle Controls.

Glioma studies using CSF-1R inhibitors demonstrated robust responses after about 1 week of treatment. Gradient echo MR imaging of brain tumors was acquired on a 4.7 Tesla MRI scanner 4-9 weeks after intracranial inoculation. Region-of-interest analyses were performed to assess tumor volumes; volume-matched pairs of mice were assigned to either treatment or control groups for survival studies. Tumor volumes (mm³) were computed on sequential MRI slices. Mice (n=15 total) can be i.v.-injected with single-dose BLZ945-αMSH-PEG-C′ dots or BLZ945, as against saline vehicle (200 μL) for 10 consecutive days, and daily weights recorded. At treatment termination, mice underwent repeat MR imaging to assess tumor volume changes. Tumor volume ratios were computed by dividing post-treatment (day 10) by pretreatment (day 0) values for individual mice and as cohort averages. Efficacy (noninferiority) was established over short-term intervals (1-2 weeks). This data compared multi-dosing and toxicology of NDCs to free drug to determine if NDC PK improves therapeutic index vs. free drug. Gliomas were isolated and dissociated resulting in a single cell suspension that can be stained with dye-labeled antibodies for flow cytometry analysis and sorting. Co-localization of particles in specific TME cell types were achieved by applying a multi-fluorochrome antibody panel (e.g., CD45, CD11b, CD11c) to identify myeloid and lymphoid cell types.

What is claimed is:

1. A method of treating cancer, the method comprising administering to a subject a pharmaceutical composition comprising a nanoparticle drug conjugate (NDC), the nanoparticle drug conjugate comprising:
a nanoparticle with average diameter no greater than 20 nm;
a linker moiety; and
a drug moiety,
wherein the drug moiety and the linker moiety form a cleavable linker-drug construct that is attached (e.g., covalently and/or non-covalently bound) to the nanoparticle, and wherein the NDC readily diffuses within tumor interstitium.

2. The method of claim 1, wherein the cancer comprises a member selected from the group consisting of a malignant brain tumor, a metastatic brain tumor, non-small cell lung carcinoma (NSCLC) and a glioblastoma multiforme (GBM).

3. The method of claim 1 or 2, wherein the method achieves sufficient drug moiety accumulation and/or (more uniform) distribution within tissue to treat a primary malignant tumor or metastatic disease.

4. The method of any one of claims 1 to 3, wherein the method achieves sufficient drug moiety accumulation and/or (more uniform) distribution within cerebrospinal fluid so as to treat leptomeningeal metastases.

5. The method of any one of claims 1 to 4, wherein the nanoparticle has an average diameter from 3 to 8 nm.

6. The method of any one of claims 1 to 5, wherein the linker moiety comprises a cleavable linker and/or a biocleavable linker.

7. The method of any one of claims 1 to 6, wherein the linker moiety comprises a member selected from the group consisting of a peptide, a hydrazone, a PEG, and a moiety comprising one or more amino acids (natural and/or non-natural amino acid).

8. The method of any one of claims 1 to 6, wherein the linker moiety comprises an enzyme sensitive linker moiety.

9. The method of any one of claims 1 to 8, wherein the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

10. The method of any one of claims 1 to 9, wherein the nanoparticle drug conjugate comprises one or more targeting moieties.

11. The method of claim 10, wherein the nanoparticle drug conjugate comprises from 1 to 20 discrete targeting moieties (e.g., of the same type or of different types).

12. The method of any one of the preceding claims, comprising administering nanoparticle drug conjugates with a first moiety for delivering and targeting the drug moiety to a tumor and NDCs with a second moiety for delivering and targeting the drug moiety to the microenvironment surrounding the tumor.

13. The method of claim 12, wherein the first and second moieties may be on the same or different NDCs that are administered to the subject in one or more compositions.

14. The method of any one of the preceding claims, wherein the NDC comprises a radioisotope.

15. The method of claim 14, wherein the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

16. The method of any one of the preceding claims, wherein the drug moiety comprises a small molecule inhibitor SMI (e.g., CSF-1R, dasatinib) or a chemotherapeutic.

17. The method of any one of the preceding claims, wherein the nanoparticle drug conjugate comprises an immunomodulator and/or anti-inflammatory agent.

18. The method of claim 17, wherein the immunomodulator and/or anti-inflammatory agent comprises αMSH.

19. The method of any one of the preceding claims, the method comprising administration (e.g., for immunotherapy) of an antibody or antibody fragment.

20. The method of claim 19, wherein the composition comprises an antibody and/or an NDC with antibody fragment attached.

21. The method of any one of the preceding claims, the method comprising administration of a NDC with antibody fragment attached, wherein the antibody fragment is a member selected from the set consisting of a recombinant antibody fragment (fAbs), a single chain variable fragment (scFv), and a single domain antibody (sdAb) fragment.

22. The method of claim 21, wherein the antibody fragment is a single chain variable fragment (scFv).

23. The method of claim 21 or 22, wherein the antibody fragment is a single domain (sdAb) fragment.

24. The method of any one of the preceding claims, wherein the pharmaceutical composition comprises nanoparticles targeted to cancer cells such that the nanoparticles accumulate in concentrations sufficient to induce ferroptosis of the cancer cells.

25. The method of any one of the preceding claims, wherein the nanoparticle comprises silica.

26. The method of any one of the preceding claims, wherein the nanoparticle comprises a silica-based core and silica shell surrounding at least a portion of the core.

27. The method of any one of the preceding claims, wherein the pharmaceutical composition comprises a carrier.

28. A method of in vivo diagnosis and/or staging of cancer, wherein the in vivo diagnosis and/or staging comprises:
delivering a pharmaceutical composition to the subject, wherein the pharmaceutical composition comprises a nanoparticle drug conjugate (NDC), the nanoparticle drug conjugate comprising:
a nanoparticle with an average diameter no greater than 20 nm;
a linker moiety;
a drug moiety, wherein the drug moiety and the linker moiety form a cleavable linker-drug construct that is attached (e.g., covalently and/or non-covalently bound) to the nanoparticle, and wherein the NDC readily diffuses within tumor interstitium; and
a radioisotope; and
detecting the radioisotope in the subject.

29. The method of claim 28, wherein the NDC comprises one or more targeting moieties.

30. The method of claim 28 or 29, wherein the cancer comprises a member selected from the group consisting of a malignant brain tumor, a metastatic brain tumor, non-small cell lung carcinoma (NSCLC) and a glioblastoma multiforme (GBM).

31. The method of any one of claims 28 to 30, wherein the method achieves sufficient drug moiety accumulation and/or (more uniform) distribution within tissue to treat a primary malignant tumor or metastatic disease.

32. The method of any one of claims 28 to 31, wherein the method achieves sufficient drug moiety accumulation and/or (more uniform) distribution within cerebrospinal fluid so as to treat leptomeningeal metastases.

33. The method of any one of claims 28 to 32, wherein the nanoparticle has an average diameter from 3 to 8 nm.

34. The method of any one of claims 28 to 33, wherein the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

35. The method of any one of claims 28 to 34, wherein the linker moiety comprises a cleavable linker and/or a biocleavable linker.

36. The method of any one of claims 28 to 35, wherein the linker moiety comprises a member selected from the group consisting of a peptide, a hydrazone, a PEG, and a moiety comprising one or more amino acids (natural and/or non-natural amino acid).

37. The method of any one of claims 28 to 35, wherein the linker moiety comprises an enzyme sensitive linker moiety.

38. The method of any one of claims 28 to 37, wherein the drug moiety comprises a member selected from the group consisting of a small molecule inhibitor (SMI), a tyrosine kinase inhibitor (TKI), an EGFR inhibitor, and a PDGFR inhibitor.

39. The method of any one of claims 28 to 38, comprising, mapping a concentration of the radioisotope in the subject, e.g., in 2D or 3D, and, optionally, detecting fluorescence from a fluorescent compound (e.g., the fluorescent compound attached to and/or incorporated within the nanoparticle of the NDC).

40. The method of claim 39, wherein the radioisotope detection/mapping step is part of a treatment of the cancer.

41. The method of claim 40, wherein the method is a theranostic method.

42. A pharmaceutical composition comprising a nanoparticle drug conjugate (NDC), the nanoparticle drug conjugate comprising:
a nanoparticle with an average diameter no greater than 20 nm;
a linker moiety;
a drug,
wherein the drug moiety and the linker moiety form a cleavable linker-drug construct that is attached (e.g., covalently and/or non-covalently bound) to the nanoparticle, and wherein the NDC readily diffuses within tumor interstitium;
for use in a method of treating cancer, the method comprising administering to a subject a pharmaceutical composition comprising the nanoparticle drug conjugate.

43. The pharmaceutical composition of claim 42, wherein the NDC comprises one or more targeting moieties.

44. The pharmaceutical composition of claim 42 or 43, wherein the NDC comprises a radioisotope.

45. The pharmaceutical composition of any one of claims 42 to 44, wherein the cancer comprises a member selected from the group consisting of a malignant brain tumor, a metastatic brain tumor, non-small cell lung carcinoma (NSCLC) and a glioblastoma multiforme (GBM).

46. The pharmaceutical composition of any one of claims 42 to 45, wherein the method of treating cancer achieves sufficient drug moiety accumulation and/or (more uniform) distribution within tissue to treat a primary malignant tumor or metastatic disease.

47. The pharmaceutical composition of any one of claims 42 to 46, wherein the method of treating cancer achieves sufficient drug moiety accumulation and/or (more uniform) distribution within cerebrospinal fluid so as to treat leptomeningeal metastases.

48. The pharmaceutical composition of any one of claims 42 to 47, wherein the nanoparticle has an average diameter from 3 to 8 nm

49. The pharmaceutical composition of any one of claims 44 to 48, wherein the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

50. The pharmaceutical composition of any one of claims 42 to 49, wherein the linker moiety comprises a cleavable linker and/or a biocleavable linker.

51. The pharmaceutical composition of any one of claims 42 to 50, wherein the linker moiety comprises a member selected from the group consisting of a peptide, a hydrazone, a PEG, and a moiety comprising one or more amino acids (natural and/or non-natural amino acid).

52. The pharmaceutical composition of any one of claims 42 to 50, wherein the linker moiety comprises an enzyme cleavable linker.

53. The pharmaceutical composition of any one of claims 42 to 52, wherein the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

54. The pharmaceutical composition of any one of claims 42 to 53, wherein the pharmaceutical composition comprises a carrier.

55. A pharmaceutical composition comprising a nanoparticle drug conjugate (NDC), the nanoparticle drug conjugate comprising:
a nanoparticle with an average diameter no greater than 20 nm;
a linker moiety;
a drug moiety, wherein the NDC readily diffuses within tumor interstitium;
for use in a method of in vivo diagnosis and/or staging of cancer, wherein the in vivo diagnosis and/or staging comprises:
delivering the composition to the subject; and
detecting the radioisotope in the subject.

56. The pharmaceutical composition of claim 55, wherein the NDC comprises one or more targeting moieties.

57. The pharmaceutical composition of claim 55 or 56, wherein the NDC comprises a radioisotope (e.g., PET tracer), e.g., ⁸⁹Zr, ⁶⁴Cu, and/or ¹²⁴I, (e.g., within the nanoparticle, attached to the nanoparticle (directly or via a linker), and/or attached to the drug moiety).

58. The pharmaceutical composition of any one of claims 55 to 57, wherein the cancer comprises a member selected from the group consisting of a malignant brain tumor, a metastatic brain tumor, non-small cell lung carcinoma (NSCLC) and a glioblastoma multiforme (GBM).

59. The pharmaceutical composition of any one of claims 55 to 58, wherein the method achieves sufficient drug moiety accumulation and/or (more uniform) distribution within tissue to treat a primary malignant tumor or metastatic disease.

60. The pharmaceutical composition of any one of claims 55 to 59, wherein the method achieves sufficient drug moiety accumulation and/or (more uniform) distribution within cerebrospinal fluid so as to treat leptomeningeal metastases.

61. The pharmaceutical composition of any one of claims 55 to 60, wherein the nanoparticle has an average diameter from 3 to 8 nm.

62. The pharmaceutical composition of any one of claims 55 to 61, wherein the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

63. The pharmaceutical composition of any one of claims 55 to 62, wherein the linker moiety comprises a cleavable linker and/or a biocleavable linker.

64. The pharmaceutical composition of any one of claims 55 to 63, wherein the linker moiety comprises a member selected from the group consisting of a peptide, a hydrazone, a PEG, and a moiety comprising one or more amino acids (natural and/or non-natural amino acid).

65. The pharmaceutical composition of any one of claims 55 to 63, wherein the linker moiety comprises an enzyme sensitive linker.

66. The pharmaceutical composition of any one of claims 55 to 65, wherein the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

67. The pharmaceutical composition of any one of claims 55 to 66, comprising, mapping a concentration of the radioisotope in the subject, e.g., in 2D or 3D, and, optionally, detecting fluorescence from a fluorescent compound (e.g., the fluorescent compound attached to and/or incorporated within the nanoparticle of the NDC).

68. The pharmaceutical composition of any one of claims 55 to 67, wherein the radioisotope detection/mapping step is part of a treatment of the cancer.

69. The pharmaceutical composition of claim 68, wherein the method is a theranostic method.

70. The pharmaceutical composition of any one of claims 55 to 69, wherein the pharmaceutical composition comprises a carrier.

71. A pharmaceutical composition comprising a nanoparticle drug conjugate (NDC), the nanoparticle drug conjugate comprising:
a nanoparticle with an average diameter no greater than 20 nm;
a linker moiety; and
a drug moiety, wherein the NDC readily diffuses within tumor interstitium.

72. The pharmaceutical composition of claim 71, wherein the NDC comprises one or more targeting moieties.

73. The pharmaceutical composition of claim 71 or 72, wherein the NDC comprises a radioisotope.

74. The pharmaceutical composition of any one of claims 71 to 73, wherein the tumor comprises a member selected from the group consisting of a malignant brain tumor, a metastatic brain tumor, non-small cell lung carcinoma (NSCLC), and a glioblastoma multiforme (GBM).

75. The pharmaceutical composition of claim 71 or 74, wherein the NDC achieves sufficient drug moiety accumulation and/or (more uniform) distribution within tissue to treat a primary malignant tumor or metastatic disease.

76. The pharmaceutical composition of any one of claims 71 to 74, wherein the NDC achieves sufficient drug moiety accumulation and/or (more uniform) distribution within cerebrospinal fluid so as to treat leptomeningeal metastases.

77. The pharmaceutical composition of any one of claims 71 to 76, wherein the nanoparticle has an average diameter from 3 to 8 nm.

78. The pharmaceutical composition of any one of claims 71 to 77, wherein the pharmaceutical composition comprises one or more members selected from the group consisting ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

79. The pharmaceutical composition of any one of claims 71 to 78, wherein the linker moiety comprises a cleavable linker and/or a biocleavable linker.

80. The pharmaceutical composition of any one of claims 71 to 79, wherein the linker moiety comprises a member selected from the group consisting of a peptide, a hydrazone, a PEG, and a moiety comprising one or more amino acids (natural and/or non-natural amino acid).

81. The pharmaceutical composition of any one of claims 71 to 79, wherein the linker moiety comprises an enzyme sensitive linker.

82. The pharmaceutical composition of any one of claims 71 to 81, wherein the drug moiety comprises a member selected from the group consisting of a small molecule inhibitor (SMI), a tyrosine kinase inhibitor (TKI), an EGFR inhibitor (e.g., gefitinib), and a PDGFR inhibitor (e.g., dasatinib).

83. A method of manipulating behavior of cells in a tumor microenvironment, the method comprising administering to a subject the pharmaceutical composition comprising a nanoparticle conjugate, the nanoparticle conjugate comprising:
a nanoparticle with an average diameter no greater than 20 nm;
a linker moiety; and
a modulator moiety, wherein the nanoparticle conjugate readily diffuses within tumor interstitium.

84. The method of claim 83, wherein the nanoparticle conjugate comprises one or more targeting moieties.

85. The method of claim 83 or 84, wherein the nanoparticle conjugate comprises a radioisotope.

86. The method of claim 85, wherein the tumor comprises a member selected from the group consisting of a malignant brain tumor, a metastatic brain tumor, non-small cell lung carcinoma (NSCLC) and a glioblastoma multiforme (GBM).

87. The method of 85 or 86, wherein the nanoparticle has an average diameter from 3 to 8 nm.

88. The method of any one of claims 85 to 87, wherein the radioisotope comprises one or more members selected from the group consisting of ^{99m}Tc, ¹¹¹In, ⁶⁴Cu, ⁶⁷Ga, ⁶⁸Ga, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, and ¹⁹²Ir.

89. The method of any one of claims 85 to 88, wherein the linker moiety comprises a cleavable linker and/or a biocleavable linker.

90. The method of any one of claims 85 to 89, wherein the linker moiety comprises a member selected from the group consisting of a peptide, a hydrazone, a PEG, and a moiety comprising one or more amino acids (natural and/or non-natural amino acid).

91. The method of any one of claims 83 to 90, wherein the linker moiety comprises an enzyme sensitive linker.

92. The method of any one of claims 83 to 91, wherein the cells comprise a member selected from the group consisting of macrophages, tumor-associated macrophages and/or microglia (TAMs), dendritic cells, and T cells.

93. The method of an one of claim 83 to 92, wherein the tumor microenvironment is in vivo, in the treatment of cancer, brain cancer, malignant cancer, and/or malignant brain cancer.

94. The method of any one of claims 83 to 93, wherein the modulator moiety comprises an inhibitor of colony stimulating factor-1 (CSF-1R), for targeting TAMs, wherein the modulator moiety and the linker moiety form a cleavable linker-modulator construct that is attached (e.g., covalently and/or non-covalently bound) to the nanoparticle.

95. The method of any one of claims 83 to 93, wherein the modular moiety comprises an immunomodulator (αMSH), wherein the modulator moiety and the linker moiety form a cleavable linker-modulator construct that is attached (e.g., covalently and/or non-covalently bound) to the nanoparticle.
